(19) United States
(12) Patent Application Publication
(10) Pub. No.: US 2026/0027235 A1
(43) Pub. Date: Jan. 29, 2026

(54) ADENO-ASSOCIATED VIRUS VECTOR DELIVERY OF MICRO-DYSTROPHIN TO TREAT MUSCULAR DYSTROPHY

(71) Applicant: RESEARCH INSTITUTE AT NATIONWIDE CHILDREN'S HOSPITAL, Columbus, OH (US)

(72) Inventors: Louise Rodino-Klapac, Columbus, OH (US); Jerry R. Mendell, Columbus, OH (US)

(73) Assignee: RESEARCH INSTITUTE AT NATIONWIDE CHILDREN'S HOSPITAL, Columbus, OH (US)

(21) Appl. No.: 19/298,798

(22) Filed: Aug. 13, 2025

Related U.S. Application Data
(63) Continuation of application No. 18/906,008, filed on Oct. 3, 2024.
Application No. 18/906,008 is a continuation of application No. 17/837,821, filed on Jun. 10, 2022, now abandoned.
Application No. 17/837,821 is a continuation of application No. 16/093,022, filed on Oct. 11, 2018, now Pat. No. 11,723,986.
(60) Provisional application No. 62/323,163, filed on Apr. 15, 2016.
(60) Provisional application No. 62/473,253, filed on Mar. 17, 2017.

(51) Int. Cl. A61K 48/00; A61K 9/00; A61K 31/7088; A61K 35/761; A61K 38/17; A61P 19/04; A61P 21/00; C07K 14/47; C12N 15/11; C12N 15/113; C12N 15/86; C12N 15/864

(52) U.S. Cl. CPC A61K 48/005; A61K 9/0019; A61K 31/7088; A61K 35/761; A61K 38/1709; A61K 38/1719; A61K 48/0058; A61K 48/0075; A61P 19/04; A61P 21/00; C07K 14/4708; C12N 15/111; C12N 15/113; C12N 15/86; C12N 15/8645; C12N 2310/141; C12N 2320/31; C12N 2330/51; C12N 2750/14141; C12N 2750/14143; C12N 2800/22; C12N 2830/008

(57) ABSTRACT

The invention provides for recombinant AAV vectors comprising a a miniaturized human micro-dystrophin gene and methods of using the recombinant vectors to reduce or prevent fibrosis in subjects suffering from muscular dystrophy.

### CROSS REFERENCE TO RELATED APPLICATIONS

This application is a continuation of U.S. patent application Ser. No. 18/906,008, filed Oct. 3, 2024, which is a continuation of U.S. patent application Ser. No. 17/837,821, filed Jun. 10, 2022, which is a continuation of U.S. patent application Ser. No. 16/093,022, filed Oct. 11, 2018, now U.S. Pat. No. 11,723,986, which is a national phase application of International Application No. PCT/US2017/027636, filed Apr. 14, 2017, which claims priority benefit of U.S. Provisional Application No. 62/323,163, filed Apr. 15, 2016 and U.S. Provisional Application No. 62/473,253, filed Mar. 17, 2017, both of which are incorporated by reference herein in their entirety.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: Filename: 140056-0796_SL.xml; Size: 67,537 bytes; created: Jan. 30, 2025.

### FIELD OF INVENTION

The invention provides gene therapy vectors, such as adeno-associated virus (AAV) vectors, expressing a miniaturized human micro-dystrophin gene and methods of using these vectors to reduce and prevent fibrosis in subjects suffering from muscular dystrophy. The invention also provides for combination gene therapy methods to protect muscle fibers from injury, increase muscle strength.

### BACKGROUND

The importance of muscle mass and strength for daily activities, such as locomotion and breathing, and for whole body metabolism is unequivocal. Deficits in muscle function produce muscular dystrophies (MDs) that are characterized by muscle weakness and wasting and have serious impacts on quality of life. The most well-characterized MDs result from mutations in genes encoding members of the dystrophin-associated protein complex (DAPC). These MDs result from membrane fragility associated with the loss of sarcolemmal-cytoskeleton tethering by the DAPC. Duchenne Muscular Dystrophy (DMD) is one of the most devastating muscle diseases affecting 1 in 5000 newborn males.

This application includes two translational approaches to develop treatment for DMD. Fibrotic infiltration is profound in DMD and is a significant impediment to any potential therapy. It is also important to consider that gene replacement alone is hampered by the severity of fibrosis, already present in very young children with DMD. In fact, muscle biopsies at the usual age of diagnosis, between 4-5 years old, show very significant levels of fibrosis.

DMD is caused by mutations in the DMD gene leading to reductions in mRNA and the absence of dystrophin, a 427 kD sarcolemmal protein associated with the dystrophin-associated protein complex (DAPC) (Hoffman et al., Cell 51(6):919-28, 1987). The DAPC is composed of multiple proteins at the muscle sarcolemma that form a structural link between the extra-cellular matrix (ECM) and the cytoskeleton via dystrophin, an actin binding protein, and alpha-dystroglycan, a laminin-binding protein. These structural links act to stabilize the muscle cell membrane during contraction and protect against contraction-induced damage. With dystrophin loss, membrane fragility results in sarcolemmal tears and an influx of calcium, triggering calcium-activated proteases and segmental fiber necrosis (Straub et al., Curr Opin. Neurol. 10(2):168-75, 1997). This uncontrolled cycle of muscle degeneration and regeneration ultimately exhausts the muscle stem cell population (Sacco et al., Cell, 2010. 143(7): p. 1059-71; Wallace et al., Annu Rev Physiol, 2009. 71: p. 37-57), resulting in progressive muscle weakness, endomysial inflammation, and fibrotic scarring.

Without membrane stabilization from dystrophin or a micro-dystrophin, DMD will manifest uncontrolled cycles of tissue injury and repair and ultimately replace lost muscle fibers with fibrotic scar tissue through connective tissue proliferation. Fibrosis is characterized by the excessive deposits of ECM matrix proteins, including collagen and elastin. ECM proteins are primarily produced from cytokines such as TGFβ that is released by activated fibroblasts responding to stress and inflammation. Although the primary pathological feature of DMD is myofiber degeneration and necrosis, fibrosis as a pathological consequence has equal repercussions. The over-production of fibrotic tissue restricts muscle regeneration and contributes to progressive muscle weakness in the DMD patient. In one study, the presence of fibrosis on initial DMD muscle biopsies was highly correlated with poor motor outcome at a 10-year follow-up (Desguerre et al., J Neuropathol Exp Neurol, 2009. 68(7): p. 762-7). These results point to fibrosis as a major contributor to DMD muscle dysfunction and highlight the need to develop therapies that reduce fibrotic tissue. Most anti-fibrotic therapies that have been tested in mdx mice act to block fibrotic cytokine signaling through inhibition of the TGFβ pathway. MicroRNAs (miRNAs) are single-stranded RNAs of ˜22 nucleotides that mediate gene silencing at the post-transcriptional level by pairing with bases within the 3′ UTR of mRNA, inhibiting translation or promoting mRNA degradation. A seed sequence of 7 bp at the 5′ end of the miRNA targets the miRNA; additional recognition is provided by the remainder of the targeted sequence, as well as its secondary structure. MiRNAs play an important role in muscle disease pathology and exhibit expression profiles that are uniquely dependent on the type of muscular dystrophy in question (Eisenberg et al. Proc Natl Acad Sci U S A, 2007. 104(43): p. 17016-21). A growing body of evidence suggests that miRNAs are involved in the fibrotic process in many organs including heart, liver, kidney, and lung (Jiang et al., Proc Natl Acad Sci U S A, 2007. 104(43): p. 17016-21). Recently, the down-regulation of miR-29 was shown to contribute to cardiac fibrosis (Cacchiarelli et al., Cell Metab, 2010. 12(4): p. 341-51) and reduced expression of miR-29 was genetically linked with human DMD patient muscles (Eisenberg et al. Proc Natl Acad Sci U S A, 2007. 104(43): p. 17016-2) . The miR-29 family consists of three family members expressed from two bicistronic miRNA clusters. MiR-29a is coexpressed with miR-29b (miR-29b-1); miR-29c is co-expressed with a second copy of miR-29b (miR-29b-2). The miR-29 family shares a conserved seed sequence and miR-29a and miR-29b each differ by only one base from miR-29c. Furthermore, electroporation of miR-29 plasmid (a cluster of miR-29a and miR-29b-1) into mdx mouse muscle reduced the expression levels of ECM components, collagen and elastin, and strongly decreased collagen deposition in muscle sections within 25 days post-treatment (Cacchiarelli et al., Cell Metab, 2010. 12(4): p. 341-51).

Adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45:555-564 (1983) as corrected by Ruffing et al., J Gen Virol, 75:3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78:6381-6388 (2004); the AAV-10 genome is provided in Mol. Ther., 13(1): 67-76 (2006); and the AAV-11 genome is provided in Virology, 330(2): 375-383 (2004). The AAVrh74 serotype is described in Rodino-Klapac et al. J. Trans. Med. 5:45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8:659-669 (1997); Kessler et al., Proc Nat. Acad Sc. USA, 93:14082-14087 (1996); and Xiao et al., J Virol, 70:8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94:5804-5809 (1997) and Murphy et al., Proc Natl Acad Sci USA, 94:13921-13926 (1997). Moreover, Lewis et al., J Virol, 76:8769-8775 (2002) demonstrated that skeletal myofibers possess the necessary cellular factors for correct antibody glycosylation, folding, and secretion, indicating that muscle is capable of stable expression of secreted protein therapeutics.

Functional improvement in patients suffering from DMD and other muscular dystrophies require both gene restoration and reduction of fibrosis. There is a need for methods of reducing fibrosis that may be paired with gene restoration methods for more effective treatments of DMD and other muscular dystrophies. miR29 is a potential gene regulator and an ideal candidate for reducing muscle fibrosis.

### SUMMARY OF INVENTION

The present invention is directed to gene therapy methods that directly reduce the three primary components of connective tissue (collagen 1, collagen 3 and fibronectin) by delivering the microRNA miR29. In this system, the miR29 binds to the 3′ UTR of the collagen and fibronectin gene to down regulate expression. The invention is directed to gene therapy vectors, e.g. AAV, expressing the guide strand of the microRNA miR29 and method of delivering miR29 to the muscle to reduce and/or prevent fibrosis.

In addition, the invention provides for combination therapies and approaches for reducing and preventing fibrosis using gene therapy vectors deliver miR-29 to suppress fibrosis along with micro-dystrophin to address the gene defect observed in DMD. As shown in Examples 5-7, the combination treatment resulted in a greater reduction in fibrosis, increased muscle size and increased muscle force.

In one embodiment, the invention provides for a rAAV vector expressing miR-29. For example, the rAAV vector comprises a polynucleotide sequence expressing miR29c such as a nucleotide sequence comprising the miR-29c target guide strand of SEQ ID NO: 3, the miR-29c guide strand of SEQ ID NO: 4 and the natural miR-30 back bone and stem loop (SEQ ID NO: 5). An exemplary polynucleotide sequence comprising the miR-29c cDNA in a miR-30 backbone is set out as SEQ ID NO: 2 (FIG. 1).

An exemplary rAAV of the invention is the pAAV.CMV.Mir29C which comprises the nucleotide sequence of SEQ ID NO: 1; wherein the CMV promoter spans nucleotides 120-526, an EF1a intron spans nucleotides 927-1087 and nucleotides 1380-1854, the guide stand of miR-29c spans nucleotide 1257-1284 and the shRNA-miR29-c with primary seed sequence spans nucleotides 1088-1375, and the poly A sequence spans nucleotides 1896-2091. In one aspect, the rAAV vectors of the invention are AAV1, AAV2, AAV4, AAV5, AAV6, AAV7, AAVrh. 74, AAV8, AAV9, AAV10, AAV11, AAV12 or AAV13.

Another exemplary rAAV of the invention is the pAAV.MHC.Mir29C which comprises the nucleotide sequence of SEQ ID NO: 12; wherein the MCK enhancer spans nucleotides 190-395, the MHC promoter spans nucleotides 396-753, an EF1a intron spans nucleotides 1155-1315 and nucleotides 1609-2083, the guide stand of miR-29c spans nucleotide 1487-1512 and the shRNA-miR29-c with primary seed sequence spans nucleotides 1316-1608, and the poly A sequence spans nucleotides 2094-2146. In one aspect, the rAAV vectors of the invention are AAV1, AAV2, AAV4, AAV5, AAV6, AAV7, AAVrh.74, AAV8, AAV9, AAV10, AAV11, AAV12 or AAV13.

In another aspect, the rAAV vectors of the invention may be operably linked to a muscle-specific control element. For example the muscle-specific control element is human skeletal actin gene element, cardiac actin gene element, myocyte-specific enhancer binding factor MEF, muscle creatine kinase (MCK), tMCK (truncated MCK), myosin heavy chain (MHC), C5-12 (synthetic promoter), murine creatine kinase enhancer element, skeletal fast-twitch troponin C gene element, slow-twitch cardiac troponin C gene element, the slow-twitch troponin I gene element, hypozia-inducible nuclear factors, steroid-inducible element or glucocorticoid response element (GRE).

For example, any of the rAAV vectors of the invention are operably linked to the muscle-specific control element comprising the MCK enhancer nucleotide sequence of SEQ ID NO: 10 and/or the MCK promoter sequence of SEQ ID NO: 11.

The invention also provides for pharmaceutical compositions (or sometimes referred to herein as simply “compositions”) comprising any of the rAAV vectors of the invention.

In another embodiment, the invention provides for methods of producing a rAAV vector particle comprising culturing a cell that has been transfected with any rAAV vector of the invention and recovering rAAV particles from the supernatant of the transfected cells. The invention also provides for viral particles comprising any of the recombinant AAV vectors of the invention.

In another embodiment, the invention provides for methods of reducing fibrosis in a subject in need comprising administering a therapeutically effective amount of any rAAV vector of the invention expressing miR-29. For example, any of the rAAV of the invention are administered to subjects suffering from muscular dystrophy to reduce fibrosis, and in particular reduces fibrosis in skeletal muscle or in cardiac muscle of the subject. These methods may further comprise the step of administering a rAAV expressing micro-dystrophin.

“Fibrosis” refers to the excessive or unregulated deposition of extracellular matrix (ECM) components and abnormal repair processes in tissues upon injury including skeletal muscle, cardiac muscle, liver, lung, kidney, and pancreas. The ECM components that are deposited include fibronectin and collagen, e.g. collagen 1, collagen 2 or collagen 3.

In another embodiment, the invention provides for methods of preventing fibrosis in a subject in need comprising administering a therapeutically effective amount of the any recombinant AAV vector of the invention expressing miR-29. For example, any of the rAAV of the invention are administered to subjects suffering from muscular dystrophy to prevent fibrosis, e.g. the rAAV of the invention expressing miR-29 are administered before fibrosis is observed in the subject. In addition, the rAAV of the invention expressing miR-29 are administered to a subject at risk of developing fibrosis, such as those suffering or diagnosed with muscular dystrophy, e.g. DMD. The rAAV of the invention are administered to the subject suffering from muscular dystrophy in order to prevent new fibrosis in these subjects. These methods may further comprise the step of administering a rAAV expressing micro-dystrophin.

The invention also provides for methods of increasing muscular force and/or muscle mass in a subject suffering from muscular dystrophy comprising administering a therapeutically effective amount of any of the rAAV vector of the invention expressing miR-29. These methods may further comprise the step of administering a rAAV expressing micro-dystrophin.

The terms “combination therapy” and “combination treatment” refer to administration of a rAAV vector of the invention expressing miR-29 and a rAAV vector expressing micro-dystrophin.

In any of the methods of the invention, the subject may be suffering from muscular dystrophy such as DMD, Becker muscular dystrophy or any other dystrophin-associated muscular dystrophy. In addition, in any of the methods of the invention, the subject may be suffering from dystrophinopathy.

In another embodiment, the invention provides for recombinant AAV vectors comprising a nucleotide sequence encoding a micro-dystrophin protein. The invention provides for a rAAV comprising a) a nucleotide sequence having at least 85% identity to the nucleotide sequence SEQ ID NO: 7 and encodes a functional micro-dystrophin protein, b) the nucleotide sequence of SEQ ID NO: 7, or c) the nucleotide sequence of SEQ ID NO: 9.

An exemplary rAAV expressing micro-dystrophin of the invention is the pAAV.mck.micro-dystrophin which comprises the nucleotide sequence of SEQ ID NO: 9 and shown in FIGS. 10 and 11. This rAAV vector comprises the MCK promoter, a chimeric intron sequence, the coding sequence for the micro-dystrophin gene, polyA, ampicillin resistance and the pGEX plasmid backbone with pBR322 origin or replication. In one aspect, the recombinant AAV vectors of the invention are AAV1, AAV2, AAV4, AAV5, AAV6, AAV7, AAVrh.74, AAV8, AAV9, AAV10, AAV11, AAV12 or AAV13.

The invention provides for rAAV vectors encoding a micro-dystrophin protein that is, e.g., at least at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% sequence identity to SEQ ID NO: 8, wherein the protein retains micro-dystrophin activity. The micro-dystrophin protein provides stability to the muscle membrane during muscle contraction, e.g. micro-dystrophin acts as a shock absorber during muscle contraction.

The invention provides for rAAV vectors expressing micro-dystrophin comprising a nucleotide sequence that has at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% sequence identity to SEQ ID NO: 7, and encodes a functional micro-dystrophin protein.

The invention provides for rAAV vectors expressing micro-dystrophin comprising a nucleotide sequence that hybridizes under stringent conditions to the nucleic acid sequence of SEQ ID NOS: 7, or compliments thereof, and encodes a functional micro-dystrophin protein.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015M sodium citrate, and 50% formamide at 42° C. See Sambrook et al., Molecular Cloning: A Laboratory Manual, 2nd Ed., Cold Spring Harbor Laboratory, (Cold Spring Harbor, N.Y. 1989). More stringent conditions (such as higher temperature, lower ionic strength, higher formamide, or other denaturing agent) may also be used, however, the rate of hybridization will be affected. In instances wherein hybridization of deoxyoligonucleotides is concerned, additional exemplary stringent hybridization conditions include washing in 6x SSC 0.05% sodium pyrophosphate at 37° C. (for 14-base oligos), 48° C. (for 17-base oligos), 55° C. (for 20-base oligos), and 60° C. (for 23-base oligos).

Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodSO4, (SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson et al., Nucleic Acid Hybridisation: A Practical Approach, Ch. 4, IRL Press Limited (Oxford, England). Hybridization conditions can be adjusted by one skilled in the art in order to accommodate these variables and allow DNAs of different sequence relatedness to form hybrids.

In another aspect, the rAAV vectors expressing micro-dystrophin comprises the coding sequence of the micro-dystrophin gene operably linked to a muscle-specific control element. For example, the muscle-specific control element is human skeletal actin gene element, cardiac actin gene element, myocyte-specific enhancer binding factor MEF, muscle creatine kinase (MCK), tMCK (truncated MCK), myosin heavy chain (MHC), C5-12 (synthetic promoter), murine creatine kinase enhancer element, skeletal fast-twitch troponin C gene element, slow-twitch cardiac troponin C gene element, the slow-twitch troponin I gene element, hypozia-inducible nuclear factors, steroid-inducible element or glucocorticoid response element (GRE).

In addition, the invention provides for rAAV vectors expressing micro-dystrophin comprising a muscle-specific control element comprising the nucleotide sequence of SEQ ID NO: 10 or SEQ ID NO: 11.

The invention also provides for pharmaceutical compositions (or sometimes referred to herein as simply “compositions”) comprising any of the rAAV vectors of the invention.

In another embodiment, the invention provides for methods of producing a rAAV vector particle comprising culturing a cell that has been transfected with any rAAV vector of the invention and recovering rAAV particles from the supernatant of the transfected cells. The invention also provides for viral particles comprising any of the recombinant AAV vectors of the invention.

The invention also provides for methods of producing a functional micro-dystrophin protein comprising infecting a host cell with a recombinant AAV vector expressing micro-dystrophin of the invention and expressing a functional micro-dystrophin protein in the host cell.

In another embodiment, the invention provides for methods of reducing fibrosis in a subject in need comprising administering a therapeutically effective amount of any rAAV vector of the invention expressing micro-dystrophin. For example, any of the rAAV of the invention are administered to subjects suffering from muscular dystrophy or dystrophinopathy to reduce fibrosis, and in particular reduces fibrosis in skeletal muscle or in cardiac muscle of the subject.

In another embodiment, the invention provides for methods of preventing fibrosis in a subject in need comprising administering a therapeutically effective amount of the any recombinant AAV vector of the invention expressing micro-dystrophin. For example, any of the rAAV of the invention are administered to subjects suffering from muscular dystrophy or dystrophinopathy to prevent fibrosis, e.g. the rAAV of the invention expressing micro-dystrophin are administered before fibrosis is observed in the subject. In addition, the rAAV of the invention expressing micro-dystrophin are administered to a subject at risk of developing fibrosis, such as those suffering or diagnosed with dystrophinopathy or muscular dystrophy, e.g. DMD or Becker muscular dystrophy. The rAAV of the invention are administered to the subject suffering from dystrophinopathy or dystrophinopathy muscular dystrophy in order to prevent new fibrosis in these subjects.

The invention also provides for methods of increasing muscular force and/or muscle mass in a subject suffering from muscular dystrophy or dystrophinopathy comprising administering a therapeutically effective amount of any of the rAAV vector of the invention expressing miR-29.

Any of the foregoing methods comprising the step of administering the rAAV expressing miR-29c of the invention may comprise a further step of administering any of the rAAV expressing the micro-dystrophin described herein. The terms “combination therapy” and “combination treatment” refer to administration of a rAAV vector of the invention expressing miR-29 and an rAAV vector expressing micro-dystrophin.

In the methods of administering an rAAV vector expressing miR-29 and an rAAV vector expressing the micro-dystrophin protein, these rAAV vectors may be administered concurrently, or administered consecutively with the rAAV vector expressing miR29 administered immediately before the rAAV expressing the micro-dystrophin protein, or administered consecutively with the rAAV vector expressing miR29 is administered immediately after the rAAV expressing the micro-dystrophin protein. Alternatively, the methods of the invention are carried out wherein the AAV vector expressing the micro-dystrophin protein is administered within about 1-5 hours or 5-12 hours or 12 to 15 hours or 15 to 24 hours after administering the rAAV expressing miR-29 or the methods of the invention are carried out wherein the AAV vector expressing the micro-dystrophin protein is administered within about 1-5 hours or 5-12 hours or 12 to 15 hours or 15 to 24 hours before administering the rAAV expressing miR-29. Alternatively, the methods of the invention are carried out wherein the AAV vector expressing the micro-dystrophin protein is administered within about 1 or 6 or 12 or 24 hours after administering the rAAV expressing miR-29 or the methods of the invention are carried out wherein the AAV vector expressing the micro-dystrophin protein is administered within about 1 or 6 or 12 or 24 hours before administering the rAAV expressing miR-29.

The invention contemplates administering any of the AAV vectors of the invention to patients diagnosed with dystrophinopathy or muscular dystrophy, such as DMD or Becker Muscular dystrophy, before fibrosis is observed in the subject or before the muscle force has been reduced in the subject or before the muscle mass has been reduced in the subject.

The invention also contemplates administering any of the rAAV of the invention to a subject suffering from dystrophinopathy or muscular dystrophy, such as DMD or Becker Muscular dystrophy, who already has developed fibrosis, in order to prevent new fibrosis in these subjects. The invention also provides for administering any of the rAAV of the invention to the patient suffering from muscular dystrophy who already has reduced muscle force or has reduced muscle mass in order to protect the muscle from further injury.

In any of the methods of the invention, the rAAV vector are administered by intramuscular injection or intravenous injection.

In addition, in any of the methods of the invention, the rAAV vector or composition is administered systemically. For examples, the rAAV vector or composition is parentally administration by injection, infusion or implantation.

In another embodiment, the invention provides for composition comprising any of the rAAV vectors expressing miR29 or any of the rAAV vectors expressing micro-dystrophin or comprising both a rAAV vector expressing miR-29 and a rAAV vector expressing micro-dystrophin for reducing fibrosis in a subject in needIn addition, the invention provides for compositions comprising any of the recombinant AAV vectors expressing miR29 or any of the rAAV vectors expressing micro-dystrophin or comprising both a rAAV vector expressing miR-29 and a rAAV vector expressing micro-dystrophin for preventing fibrosis in a patient suffering from dystrophinopathy or muscular dystrophy, such as DMD or Becker Muscular dystrophy.

The invention also provides for compositions comprising any of the rAAV vectors of the invention expressing miR29 or any of the rAAV vectors expressing micro-dystrophin protein or comprising both a rAAV vector expressing miR-29 and a rAAV vector expressing micro-dystrophin protein for increasing muscular force and/or muscle mass in a subject suffering from dystrophinopathy or muscular dystrophy, such as DMD or Becker Muscular dystrophy.

In a further embodiment, the invention provides for compositions comprising any of the rAAV vectors of the invention expressing miR29 or any of the rAAV vectors expressing micro-dystrophin protein or comprising both a rAAV vector expressing miR-29 and a rAAV vector expressing micro-dystrophin protein for treatment of dystrophinopathy or muscular dystrophy, such as DMD or Becker Muscular dystrophy.

The compositions of the invention are formulated for intramuscular injection or intravenous injection. The composition of the invention is also formulated for systemic administration, such as parentally administration by injection, infusion or implantation. In addition, any of the compositions are formulated for administration to a subject suffering from dystrophinopathy or muscular dystrophy, such as DMD, Becker muscular dystrophy or any other dystrophin associated muscular dystrophy.

In a further embodiment, the invention provides for use of any of the rAAV vectors of the invention expressing miR29 or any of the rAAV vectors expressing micro-dystrophin or comprising both a rAAV vector expressing miR-29 and a rAAV vector expressing micro-dystrophin for preparation of a medicament for reducing fibrosis in a subject in need. For example, the subject is in need suffering from dystrophinopathy or muscular dystrophy, such as DMD, Becker muscular dystrophy or any other dystrophin associated muscular dystrophy.

In another embodiment, the invention provides for provides for use of any of the rAAV vectors of the invention expressing miR29 or any of the rAAV vectors expressing micro-dystrophin or comprising both a rAAV vector expressing miR-29 and a rAAV vector expressing micro-dystrophin for the preparation of a medicament for preventing fibrosis in a subject suffering from muscular dystrophy. In addition, the invention provides for use of the recombinant AAV vectors of the invention expressing miR29 or any of the rAAV vectors expressing micro-dystrophin or comprising both a rAAV vector expressing miR-29 and a rAAV vector expressing micro-dystrophin for the preparation of a medicament for the increasing muscular strength and/or muscle mass in a subject suffering from dystrophinopathy or muscular dystrophy, such as DMD or Becker Muscular dystrophy.

The invention contemplates use of the any of the AAV vectors of the invention for the preparation of a medicament for administration to a patient diagnosed with DMD before fibrosis is observed in the subject or before the muscle force has been reduced in the subject or before the muscle mass has been reduced in the subject.

The invention also contemplates use of any of the AAV vectors of the invention for the preparation of a medicament for administration to administering any of the rAAV of the invention to a subject suffering from muscular dystrophy who already has developed fibrosis, in order to prevent new fibrosis in these subjects. The invention also provides for administering any of the rAAV of the invention to the patient suffering from muscular dystrophy who already has reduced muscle force or has reduced muscle mass in order to protect the muscle from further injury.

The invention also provides for use of the rAAV vectors of the invention expressing miR296 or any of the rAAV vectors expressing micro-dystrophin or comprising both a rAAV vector expressing miR-29 and a rAAV vector expressing micro-dystrophin for the preparation of a medicament for treatment of muscular dystrophy.

In any of the uses of the invention, the medicament is formulated for intramuscular injection. In addition, any of the medicaments may be prepared for administration to a subject suffering from muscular dystrophy such as DMD or any other dystrophin associated muscular dystrophy.

In addition, any of the medicaments of the invention may be a combination therapy in which the rAAV vectors expressing miR-29 and rAAV vectors expressing micro-dystrophin are administered concurrently, or administered consecutively with the rAAV vector expressing miR29 administered immediately before the rAAV expressing micro-dystrophin, or administered consecutively with the rAAV vector expressing miR29 administered immediately after the rAAV expressing micro-dystrophin. Alternatively, the medicament comprises administration of the AAV vector expressing micro-dystrophin administered within about 1-5 hours after administering the rAAV expressing miR-29 or the medicament comprises the AAV vector expressing micro-dystrophin administered within about 1-5 hours before administering the rAAV expressing miR-29.

### BRIEF DESCRIPTION OF DRAWING

FIG. 1 provide a schematic of rAAV vector scAA VCrh.74.CMV.miR29c and the nucleotide sequence of the miR-29c in a natural miR-30 backbone and the nucleotide sequence of the predicted hairpin structure. Figure discloses SEQ ID NOS 2, 5, 3-4, 6 and 13-15, respectively, in order of appearance.

FIGS. 2A-2C illustrate that injection of miR-29c into muscle reduces collagen throughout the muscle and restores miR-29c expression.

FIGS. 3A-3C demonstrate that injection of miR-29c improves absolute muscle force (panel A) and specific muscle force (panel B) but does not protect against contraction-induced damage (panel C).

FIGS. 4A-4C display the number of muscle fibers expression micro-dystrophin to measure of efficacy of transgene delivery.

FIGS. 5A-5C demonstrate that co-delivery of miR-29c with micro-dystrophin reduces collagen expression (panel A) and fibrosis-induced dystrophin expression.

FIGS. 6A-6D illustrate that intramuscular injection of miR-29c/micro-dystrophin inhibits extracellular matrix (ECM) in mdx/utrn^{+/-} mice as measured by collagen 1 alpha (panel A), collagen 3 alpha (panel B), fibronectin (panel C) and TGF-β (panel D).

FIGS. 7A-7C demonstrate the intramuscular injection of miR-29c increased absolute force (panel A), normalized specific force (panel B) and added protection from contraction-induce damage (panel C) in the muscle.

FIG. 8 illustrates that the miR-29c/μ-dys combination increases muscle size in mice treated at 3 months of age. Sections of treated and untreated mdx/utrn^{+/-} gastrocnemius muscles stained with picrosirius Red to stain for collagen are shown. Fibrotic areas are pink and intact muscle is in green. On the macroscopic level, miR-29c/μ-dys combination decreases fibrosis and increases total cross sectional area.

FIGS. 9A-9F demonstrate that treatment with miR-29c co-delivered with micro-dystrophin increased muscle hypertrophy and hyperplasia as shown by an increase in the overall weight of the injected gastroc compared to either one injected alone (panel A), an increase in the an increase in average fiber size (panel B), an increase in cross-sectional area of the muscle (panel D; uninjected: 24.6 vs. miR-29c: 26.3 vs. micro-dys: 26.6 vs. micro-dys/miR-29c: 33.1) and an increase in the number of muscle fibers (panel E) but the number of muscle fibers per unit area was not affected (panel F). Panel C compares mdx/utrn^{+/-} controls with miR-29c/μ-dys treated mdx/utrn^{+/-}, the average diameter increased from 25.96 to 30.97 μm

FIGS. 10A-10G demonstrate that early treatment of AAV.miR-29c/micro-dystrophin combination therapy is more effective at reducing fibrosis and ECM expression. Panel A shows picrosirius red staining of wild-type, uninjected, AAV.miR-29c, AAV.micro-dystrophin, and AAV.miR-29c/AAV.micro-dystrophin of mice injected at 4-5 wks of age taken out twelve weeks post-injection. Panel B provides quantification of picrosirius red staining showing co-treated muscle had a 51.1% reduction in collagen compared to uninjected GAS muscle. Panel C demonstrates that qRT-PCR confirms an increase in miR-29c transcript levels in the treated cohorts. Semi-quantitative qRT-PCR shows a significant reduction in collagen I and III (panels d, e), fbn (panel f) and TGF-β1 (panel g) levels in the AAV.miR-29c/AAV.micro-dystrophin treated muscle compared to the contralateral limb and each of the single therapies Error bars, SEM for n=5 (scAAVrh.74.CMV.miR-29c) , n=5 (scAAVrh.74.CMV.miR-29c/ssAAVrh.74.MCK.micro-dystrophin), n=6 (ssAA Vrh. 74.MCK.micro-dystrophin), n=9(mdx/utrn^{+/-} mice). 1-way ANOVA (*p<0.05, ** p<0.01, *** p<0.001)

FIG. 11 demonstrates early combination therapy restores force and protects against contraction-induced damage. Measurement of absolute (panel A) and normalized specific force (panel b) following tetanic contraction in all three treatment injected GAS muscles were significantly increased compared to untreated mdx/utrn^{+/-} muscle (panel C). Muscles were then assessed for loss of force following repetitive eccentric contractions. Only mice co-treated with miR-29c/micro-dystrophin and micro-dystrophin alone showed a protection from loss of force compared with untreated mdx/utrn^{+/-} muscles (blue). Two-way analysis of variance demonstrates significance in decay curves Error bars, SEM for n=5 (rAAVrh.74.CMV.miR-29c), n=6 (rAAVrh.74.CMV.miR-29c/rAAVrh.74.MCK.micro-dystrophin), n=5 (rAAVrh.74.MCK.micro-dystrophin), n=15 (mdx/utrn+/− mice). 1-way ANOVA (*p<0.05, **p<0.01, ***p<0.001, ****p<0.0001).

FIG. 12 illustrates miR-29c/micro-dystrophin combination treatment increases muscle size in mice treated at 1 month of age. Treated and untreated mdx/utrn^{+/-} GAS muscles were sectioned and staining with picrosirius Red to stain for collagen. Fibrotic areas are pink and intact muscle is in green. On the macroscopic level, miR-29c/micro-dystrophin combination decreases fibrosis and increases total cross sectional area.

FIGS. 13A-13G demonstrate that early treatment (at 4-5 weeks) of AAV.MCK.miR-29c/micro-dystrophin combination therapy is more effective at reducing fibrosis and ECM expression. Panel A provide picrosirius red staining of uninjected and AAV.MCK.miR-29c/AAV.MCK.micro-dystrophin of mice injected at 4-5wks of age taken out twelve weeks post-injection. Original magnification, x20 Panel B provides quantification of picrosirius red staining demonstrating co-treated muscle had a 50.9% reduction in collagen compared to untreated GAS muscle. Panel C provides qRT-PCR confirming an increase in miR-29c transcript levels in the treated cohort. Semi-quantitative qRT-PCR shows a significant reduction in Collagen 1A (Col1A; panel D) and Collagen 3A (Col3A; panel E), Fibronectin (Fbn; panel F) and Tgfβ1 (panel G) levels in the AAV.MCK.miR- 29c/AAV.micro-dystrophin treated muscle compared to the contralateral limb therapies. (*p<0.05, ****p<0.0001).

FIGS. 14A-14G demonstrate that late treatment (treatment at 12 weeks) with AAV.MCK.miR-29c/micro-dystrophin combination therapy is effective at reducing fibrosis and ECM expression. Panel A provides picrosirius red staining of untreated, AAV.MCK.miR-29c and AAV.MCK.miR-29c/AAV.micro-dystrophin twelve weeks post-injection. Original magnification, x20. Panel B provides quantification of picrosirius red staining which demonstrates that co-treated muscle had a 30.3% reduction in collagen compared to untreated GAS muscle. Panel C provides qRT-PCR confirming an increase in miR-29c transcript levels in the treated cohorts. Semi-quantitative qRT-PCR demonstrated a significant reduction in Collagen 1A (Col1A; panel D), Collagen 3A (Col3A; panel E), Fibronectin (Fbn; Panel F) and TgfB1 (panel G) levels in the AAV.miR-29c/AAV.micro-dystrophin treated muscle compared to the contralateral limb. One-way ANOVA. All data represent mean±SEM. (**p<0.01, ****p<0.0001).

FIGS. 15A-15C demonstrate that early combination therapy (treatment at 4-5 weeks) restored force and protected against contraction-induced damage. Measurement of absolute (panel A) and normalized specific force (panel B) following tetanic contraction MCK.miR-29c/micro-dystrophin injected GAS muscles were significantly increased compared to untreated mdx/utrn^{+/-} muscle. (C) Muscles were then assessed for loss of force following repetitive eccentric contractions. Mice co-treated with miR-29c/micro-dystrophin and micro-dystrophin alone showed protection from loss of force compared with untreated mdx/utrn^{+/-} muscles (red). Two-way ANOVA. All data represent mean±SEM (****p<0.0001).

FIGS. 16A-16C demonstrate that late combination therapy restored force and protected against contraction-induced damage. Measurement of absolute (panel A) and normalized specific force (panel B) following tetanic contraction rAAV.MCK.miR-29c and rAAV expressing micro-dystrophin injected GAS muscles were significantly increased compared to untreated mdx/utrn^{+/-} muscle. In Panel C, muscles were then assessed for loss of force following repetitive eccentric contractions. Mice co-treated with rAAV.MCK.miR-29c/rAAV expressing micro-dystrophin showed a protection from loss of force compared with untreated mdx/utrn^{+/-} muscles (red). Two-way ANOVA. All data represent mean±SEM (**p<0.01, ****p<0.0001).

FIGS. 17A-17D demonstrate that combination treatment increases muscle hypertrophy 3 months post injection. Panel A demonstrates that rAAV. MCK.miR-29c co-delivered with rAAV expressing micro-dystrophin failed to increase the overall weight of the injected GAS. Panel B demonstrates that rAAV.MCK.miR-29c/rAAV expressing micro-dystrophin combination treatment induced an increase in average fiber size. Comparing mdx/utrn-controls with miR-29c/micro-dystrophin treated mdx/utrn^{+/-}, the average diameter increased from 28.96 to 36.03 μm. Panel C shows that co-delivery produced a shift towards wild-type fiber size distribution. Panel D provided the number of muscle fibers per mm² in the miR-29c/micro-dystrophin combination treatment was significantly less than untreated mice and wild-type (***p<0.01, ****p<0.0001).

FIGS. 18A-18B provide the nucleic acid sequence (SEQ ID NO: 1 pAAV.CMV.Mir29C) of an exemplary rAAV vector comprising the mature guide strand of miR-29c (nucleotides 1257-1284) and the natural mi-30 backbone (nucleotides 1088-1375). The construct also comprises the CMV promoter (nucleotides 120-526), two EF1a introns at nucleotides 927-1087 and 1380-1854 and a polA at nucleotides 1896-2091.

FIG. 19 provides a schematic of the rAAV vector pAAV.MCK.micro-dystrophin.

FIGS. 20A-20D provide the nucleic acid sequence (SEQ ID NO: 9; pAAV.MCK.micro-dystrophin) of an exemplary rAAV vector expressing micro-dystrophin.

FIGS. 21A-21C provide the nucleotide sequence of the human micro-dystrophin nucleotide sequence (SEQ ID NO: 7)

FIGS. 22A-22C provide the nucleotide sequence (SEQ ID NO: 12 pAAV.MCK.Mir29C) of an exemplary rAAV vector comprising the mature guide strand of miR-29c (nucleotides 1487-1512) and the natural mi-30 backbone (nucleotides 1088-1375). The construct also comprises the MCK enhancer (nucleotides 190-395), MCK promoter (nucleotides 396-753), two EF1a introns at nucleotides 1155-1315 and 1609-2083 and a polA at nucleotides 2094-2148.

### DETAILED DESCRIPTION

The present invention provides for gene therapy vectors, e.g. rAAV vectors, overexpressing miR-29 microRNA and methods of reducing and preventing fibrosis in muscular dystrophy patients. The present invention also provides for combination gene therapy methods which comprise administering a gene therapy vector expressing miR-29 in combination with a gene therapy vector expressing micro-dystrophin that is deleted in DMD patients.

Muscle biopsies taken at the earliest age of diagnosis of DMD reveal prominent connective tissue proliferation. Muscle fibrosis is deleterious in multiple ways. It reduces normal transit of endomysial nutrients through connective tissue barriers, reduces the blood flow and deprives muscle of vascular-derived nutritional constituents, and functionally contributes to early loss of ambulation through limb contractures. Over time, treatment challenges multiply as a result of marked fibrosis in muscle. This can be observed in muscle biopsies comparing connective tissue proliferation at successive time points. The process continues to exacerbate leading to loss of ambulation and accelerating out of control, especially in wheelchair-dependent patients.

Without a parallel approach to reduce fibrosis it is unlikely that the benefits of exon skipping, stop-codon read-through, or gene replacement therapies can ever be fully achieved. Even small molecules or protein replacement strategies are likely to fail without an approach to reduce muscle fibrosis. Previous work in aged mdx mice with existing fibrosis treated with AAV.micro-dystrophin demonstrated that we could not achieve full functional restoration (Human molecular genetics 22, 4929-4937 (2013)). It is also known that progression of DMD cardiomyopathy is accompanied by scarring and fibrosis in the ventricular wall. Micro-RNA delivery is particularly innovative because of lack of immune barriers and relative ease of delivery. Micro-RNAs are small (˜200 bp) and can therefore be packaged in AAV along with a therapeutic cassette to correct or bypass the genetic defect.

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

### AAV

Recombinant AAV genomes of the invention comprise nucleic acid molecule of the invention and one or more AAV ITRs flanking a nucleic acid molecule. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12 and AAV-13.Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art. To promote skeletal muscle specific expression, AAV1, AAV6, AAV8 or AAVrh.74 may be used.

DNA plasmids of the invention comprise rAAV genomes of the invention. The DNA plasmids are transferred to cells permissible for infection with a helper virus of AAV (e.g., adenovirus, E1-deleted adenovirus or herpes virus) for assembly of the rAAV genome into infectious viral particles. Techniques to produce rAAV particles, in which an AAV genome to be packaged, rep and cap genes, and helper virus functions are provided to a cell, are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAVrh.74, AAV-8, AAV-9, AAV-10, AAV-11, AAV-12 and AAV-13. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Patent. No. 5,786,211; U.S. Pat. No. 5,871,982; and U.S. Pat. No. 6,258,595. The foregoing documents are hereby incorporated by reference in their entirety herein, with particular emphasis on those sections of the documents relating to rAAV production.

The invention thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV (i.e., infectious encapsidated rAAV particles) of the invention comprise a rAAV genome. In exemplary embodiments, the genomes of both rAAV lack AAV rep and cap DNA, that is, there is no AAV rep or cap DNA between the ITRs of the genomes. Examples of rAAV that may be constructed to comprise the nucleic acid molecules of the invention are set out in International Patent Application No. PCT/US2012/047999 (WO 2013/016352) incorporated by reference herein in its entirety.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In another embodiment, the invention contemplates compositions comprising rAAV of the present invention. Compositions of the invention comprise rAAV and a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as Tween, pluronics or polyethylene glycol (PEG).

Titers of rAAV to be administered in methods of the invention will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg).

Methods of transducing a target cell with rAAV, in vivo or in vitro, are contemplated by the invention. The in vivo methods comprise the step of administering an effective dose, or effective multiple doses, of a composition comprising a rAAV of the invention to an animal (including a human being) in need thereof. If the dose is administered prior to development of a disorder/disease, the administration is prophylactic. If the dose is administered after the development of a disorder/disease, the administration is therapeutic. In embodiments of the invention, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the invention is FSHD.

Combination therapies are also contemplated by the invention. Combination as used herein includes both simultaneous treatment and sequential treatments. Combinations of methods of the invention with standard medical treatments (e.g., corticosteroids) are specifically contemplated, as are combinations with novel therapies.

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the invention may be chosen and/or matched by those skilled in the art taking into account the infection and/or disease state being treated and the target cells/tissue(s) that are to express the miR-29 miRNA and/or micro-dystrophin.

The invention provides for local administration and systemic administration of an effective dose of rAAV and compositions of the invention including combination therapy of the invention. For example, systemic administration is administration into the circulatory system so that the entire body is affected. Systemic administration includes enteral administration such as absorption through the gastrointestinal tract and parental administration through injection, infusion or implantation.

In particular, actual administration of rAAV of the present invention may be accomplished by using any physical method that will transport the rAAV recombinant vector into the target tissue of an animal. Administration according to the invention includes, but is not limited to, injection into muscle, the bloodstream and/or directly into the liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The rAAV can be used with any pharmaceutically acceptable carrier for ease of administration and handling.

The dose of rAAV to be administered in methods disclosed herein will vary depending, for example, on the particular rAAV, the mode of administration, the treatment goal, the individual, and the cell type(s) being targeted, and may be determined by methods standard in the art. Titers of each rAAV administered may range from about 1×10⁶, about 1×107, about 1×108, about 1×109, about 1×1010, about 1×1011, about 1×1012, about 1×1013, about 1×1014, or to about 1×1015 or more DNase resistant particles (DRP) per ml. Dosages may also be expressed in units of viral genomes (vg) (i.e., 1×10⁷ vg, 1×10⁸ vg, 1×10⁹ vg, 1×10¹⁰ vg, 1×10¹¹ vg, 1×10¹² vg, 1×10¹³ vg, 1×10¹⁴ vg, 1×10¹⁵ respectively). Dosages may also be expressed in units of viral genomes (vg) per kilogram (kg) of bodyweight (i.e., 1×10¹⁰ vg/kg, 1×10¹¹ vg/kg, 1×10¹² vg/kg, 1×10¹³ vg/kg, 1×10¹⁴ vg/kg, 1×10¹⁵ vg/kg respectively). Methods for titering AAV are described in Clark et al., Hum. Gene Ther., 10:1031-1039 (1999).

In particular, actual administration of rAAV of the present invention may be accomplished by using any physical method that will transport the rAAV recombinant vector into the target tissue of an animal. Administration according to the invention includes, but is not limited to, injection into muscle, the bloodstream and/or directly into the liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The rAAV can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical carriers, diluents or excipients suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction with rAAV may also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining rAAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV of the invention results in sustained expression of miR-29 or micro-dystrophin. The present invention thus provides methods of administering/delivering rAAV which express of miR-29 and or micro-dystrophin to an animal, preferably a human being. These methods include transducing tissues (including, but not limited to, tissues such as muscle, organs such as liver and brain, and glands such as salivary glands) with one or more rAAV of the present invention. Transduction may be carried out with gene cassettes comprising tissue specific control elements. For example, one embodiment of the invention provides methods of transducing muscle cells and muscle tissues directed by muscle specific control elements, including, but not limited to, those derived from the actin and myosin gene families, such as from the myoD gene family [See Weintraub et al., Science, 251:761-766 (1991)], the myocyte-specific enhancer binding factor MEF-2 [Cserjesi and Olson, Mol Cell Biol 11:4854-4862 (1991)], control elements derived from the human skeletal actin gene [Muscat et al., Mol Cell Biol, 7:4089-4099 (1987)], the cardiac actin gene, muscle creatine kinase sequence elements [See Johnson et al., Mol Cell Biol, 9:3393-3399 (1989)] and the murine creatine kinase enhancer (mCK) element, control elements derived from the skeletal fast-twitch troponin C gene, the slow-twitch cardiac troponin C gene and the slow-twitch troponin I gene: hypoxia-inducible nuclear factors (Semenza et al., Proc Natl Acad Sci USA, 88:5680-5684 (1991)), steroid-inducible elements and promoters including the glucocorticoid response element (GRE) (See Mader and White, Proc. Natl. Acad. Sci. USA 90:5603-5607 (1993)), and other control elements.

Muscle tissue is an attractive target for in vivo DNA delivery, because it is not a vital organ and is easy to access. The invention contemplates sustained expression of miRNAs from transduced myofibers.

By “muscle cell” or “muscle tissue” is meant a cell or group of cells derived from muscle of any kind (for example, skeletal muscle and smooth muscle, e.g. from the digestive tract, urinary bladder, blood vessels or cardiac tissue). Such muscle cells may be differentiated or undifferentiated, such as myoblasts, myocytes, myotubes, cardiomyocytes and cardiomyoblasts.

The term “transduction” is used to refer to the administration/delivery of the miiR29 guide strand or the coding region of the micro-dystrophin to a recipient cell either in vivo or in vitro, via a replication-deficient rAAV of the invention resulting in expression of a miR29 or micro-dystrophin by the recipient cell.

Thus, the invention provides methods of administering an effective dose (or doses, administered essentially simultaneously or doses given at intervals) of rAAV that encode miR29 and/or micro-dystrophin to a patient in need thereof.

### EXAMPLES

### Example 1 Confirmation of Duchenne Muscular Dystrophy Models

The mdx mouse provides a convenient, yet incomplete, animal model to study DMD pathogenesis. This model is a cross of the mdx mouse with a heterozygous knockout of the utrophin gene (mdx:utrn+/−), which presents with increased fibrosis and more faithfully recapitulates the pathology of human DMD. Mdx mice have a nonsense mutation in exon 23 of DMD that results in a relatively mild phenotype and a near-normal life span. By 3 weeks of age, the diaphragm and limb muscle of mdx mice develop signs of endomysial inflammation. These symptoms subside in the limb muscle after the mice reach adulthood while the inflammation in the diaphragm muscle continues to progressively worsen. In mdx mice lacking telomerase, muscular dystrophy progressively worsens with age; mdx mice lacking utrophin (DKO) have a phenotype more characteristic of human DMD with early onset muscle weakness, severe fibrosis, and premature death. Utrophin, an autosomal paralog of the dystrophin, shares a high degree of sequence homology that may compensate for the lack of dystrophin in the mdx mouse in the double KO (dystrophin plus utrophin); a severe phenotype with early death is observed. The premature death in the DKO mouse precludes progression of inflammation and fibrosis, but the mdx:utrn^{+/-} mouse presents a model with similarities to the human disease exhibiting a striking degree of fibrosis, and a longer survival than the DKO, providing a better model for our proposed translational studies. A recent report confirms the use of the mdx:utrn^{+/-} mouse as an ideal model to study fibrosis in the context of DMD. In the present study, increased fibrosis as measured by Sirius red staining was accompanied by increased collagen transcript levels and decreased mir29c levels.

### Example 2 Delivery of miR29 to DMD Mice Reduces Fibrosis

Preliminary studies have demonstrated that there is a significant increase in Sirius Red staining for collagen and a decrease in miR-29c levels in human DMD patients and the mdx/utrn^{+/-} mouse. Gene delivery of miR-29 using muscle specific AAV vectors is potentially safe and efficient. To generate the rAAV vector, referred to herein as rAAVrh.74.CMV.miR29c, the 22 nucleotide miR29c sequence (target strand SEQ ID NO: 3 and guide strand SEQ ID NO: 4) was cloned into a miR-30 scaffold driven by a CMV promoter. The expression cassette (SEQ ID NO: 2) was cloned into a self-complementary AAV plasmid and packaged using AAVrh.74, a serotype known to express well in muscle. The miR-29c cDNA was synthesized using a custom primer containing the miR-29c target (sense) strand, miR-30 stem loop and miR-29c guide (antisense) strand in the miR-30 backbone. Three bases of the miR-29c sequence were modified. This sequence was then cloned into a self-complementary AAV ITR containing plasmid driven by the CMV promoter and polyA sequence.

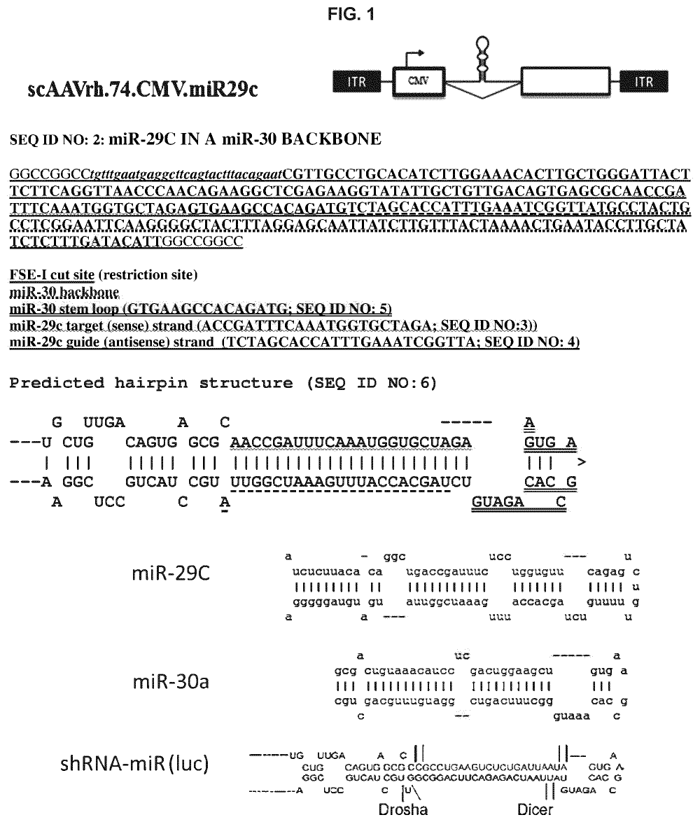

As shown in FIG. 1, the pAAV.CMV.miR29C plasmid contains the mir29c cDNA in a miR-30 stem loop backbone flanked by AAV2 inverted terminal repeat sequences (ITR). It is this sequence that was encapsidated into AAVrh.74 virions. In addition, a few nucleotides with in the miR-29c target sequence were changed to mimic Watson-crick pairing at this site as in shRNA-miR(luc). According to ShRNA-luc design, the hairpin should be perfectly complementary throughout its length. Plus, the more changes to the passenger strand, the more likely the elimination of any endogenous mechanism that regulates miR-29 processing that could recognize the miRNA via the stem. The 19th base of the guide strand was modified to a cytosine to mimic the nucleotide that precedes the cleavage site in natural mi-29c sequence and the corresponding base on the other strand was changed to preserve pairing.

The gene therapy vector scrAAVrh.74.CMV.miR29c (1×10¹¹ vgs) was injected into the quadriceps muscle of 3 month old mdx/utrn^{+/-} miceQuadriceps muscle was analyzed 3 months post-injection by Sirius Red staining and analyzed by NIH ImageJ software as described in Nevo et al. (PloS One, 6: e18049 (2011). MiR29c, collagen and elastin levels were quantified by RT-PCR. Delivery of miR-29c to young mdx/utrn^{+/-} mice significantly increases mir-29c levels and a significant reduction in Sirius red staining in the quadriceps muscle of 6 month old mdx/utrn^{+/-} mice (3 months post injection). There was a reduction in collagen and elastin levels in the treated muscles when evaluated by RT-PCR.

Demonstration of increased fibrosis and decreased miR29 expression in the mdx/utrn^{+/-} mice and dystrophin-deficient patients validates the mouse model as being representative of the human disease. Initial results using AAV-delivered miR29 as an anti-fibrotic therapy suggest that there is significant beneficial effect with reduction in Sirius Red staining and collagen and elastin levels, which are key contributors in fibrosis.

### Example 3 Injection of MiR-29c Reduces Collagen and Restores miR-29c

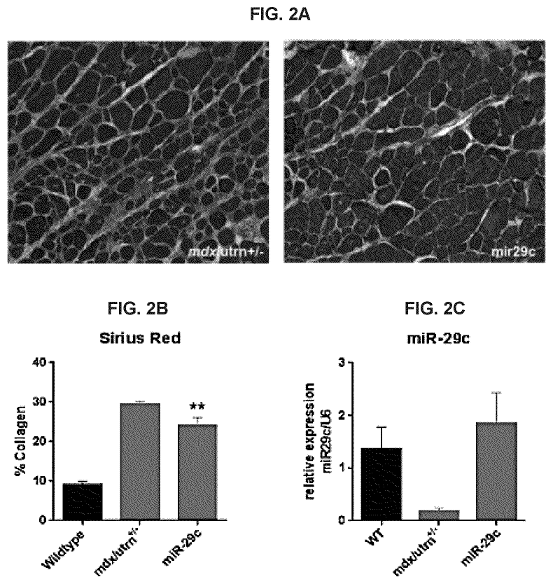

To determine whether rAAVrh. 74.CMV.MIR-29c could reduce fibrosis, 12-week-old mdx/utrn^{+/-} mice received an intramuscular injection of rAAVrh.74.CMV.MIR-29c at 5×10¹¹ vgs to the left gastrocnemius (GAS) muscle. The mice were analyzed at 12 weeks post injection. Picrosirius red staining revealed a significant decrease in collagen staining throughout the GAS muscles (FIG. 2a) compared to the untreated contralateral mdx/utrn +/− GAS muscle. Quantification of the picrosirius red staining shows that treated muscle had a 18.3% reduction in collagen compared to the untreated muscle (treated-23.3%±1.3 vs. untreated-29.5%±0.7)(FIG. 2b). To confirm overexpression of miR-29c in treated muscle, total RNA was extracted from the GAS muscle from 24 week old WT, miR-29c treated and mdx/utrn-mice and subjected to quantitative reverse-transcription—PCR (qRT-PCR) analysis for miR-29c expression. The results showed that miR-29c was significantly increased in the GAS muscle of the treated mice compared to untreated mice (FIG. 2d).

### Example 4 MiR-29c Improves Absolute and Specific Muscle Force but does not protect against Contraction-Induced Damage

Knowing that fibrosis can impact muscle function, we wanted to test whether reducing fibrosis by increasing expression of MiR-29c could protect mdx/utrn^{+/-} muscle from contraction-induced injury and increase overall force. The functional properties of the gastrocnemius muscle from mdx/utrn^{+/-} mice treated with rAAVrh.74.CMV.MIR-29c were assessed. Twelve weeks post injection, the GAS was isolated to perform in vivo force measurements.

The GAS procedure follows the protocol listed in Hakim et al., (Methods Mol Biol. 709:75-89, 2011) for analyzing transverse abdominal muscle physiology but adapted for the GAS. Briefly, mice were anesthetized using ketamine/xylazine mixture. The hind limb skin was removed to expose the GAS muscle and the Achilles tendon. The distal tendon was dissected out and a double square knot was tied around the tendon with 4-0 suture as close to the muscle as possible, another second double square knot is tied right next to the first knot and then tendon is cut. The exposed muscle was constantly dampened with saline. Mice were then transferred to a thermal controlled platform and maintained at 37°. The knee was secured to the platform with a needle through the patella tendon, the tendon suture to the level arm of the force transducer (Aurora Scientific, Aurora, ON, Canada), and the foot was secured with tape. The GAS muscle contractions were elicited by stimulating the sciatic nerve via bipolar platinum electrodes. Once the muscle was stabilized, the optimal length was determined by incremental stretching the muscle until the maximum twitch force was achieved. After a 3-minute rest period, the GAS was stimulated at 50,100,150, and 200 Hz, allowing a 1-minute rest period between each stimulus to determine maximum tetanic force. Muscle length was measured. Following a 5-minute rest, the susceptibility of the GAS muscle to contraction-induced damage was assessed. After 500 ms of stimulation, the muscle was lengthened by 10% of the optimal length. This consisted of stimulating the muscle at 150Hz for 700 ms. After the stimulation, the muscle was returned to the optimal length. The cycle was repeated every minute for a total of 5 cycles. Specific force was calculated by dividing the maximum tetanic force by the GAS muscle cross sectional area. After the eccentric contractions, the mice were then euthanized and the GAS muscle was dissected out, weighed and frozen for analysis.

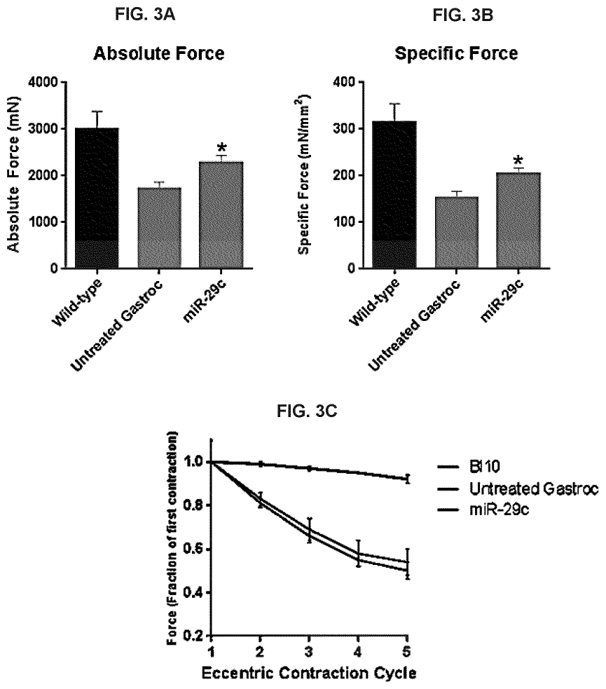

Each GAS was subjected to a series of repeated eccentric contraction. By comparing the force ratio of each contraction versus the first contraction revealed that after the fifth contraction untreated muscle decayed to 0.56±0.05 versus treated 0.50±0.04 (p≤0.0001). The injected group showed a slight decrease in the degree of protection compared to WT controls, that decayed to 0.92±0.02 (FIG. 3c). This data shows that reducing fibrosis by increasing expression of miR-29c leads to increase in both absolute and specific force but does not significantly protect muscle from contraction-induced injury.

rAAVrh.74.MiR-29c treated GAS muscle showed significant improvement in absolute force when compared to untreated mdx/utrn^{+/-} GAS muscle (rAAV.miR-29c-2277±161.7 vs. mdx/utrn^{+/-} untreated-1722±145.7; FIG. 3a), and also normalized specific force in rAAVrh.74.miR-29c treated GAS muscle specific improvement when compared to untreated GAS muscle (rAAV.miR-29c-204.7±11.7 vs. mdx/utrn^{+/-} untreated-151.6±14.5; FIG. 3b). Force was still significantly reduced when compared to wild-type controls (rAAV.miR-29c-204.7±11.7 vs. wild-type-312.0±34.1).

### Example 5 Co-delivery with Micro-Dystrophin Further Reduces Fibrosis

To determine whether miR-29c/micro-dystrophin combined gene therapy approach would be more beneficial at reducing fibrosis, 12-week-old mdx/utrn^{+/-} mice received an intramuscular injection of rAAVrh.74.CMV.MiR-29c at 5×10¹¹ vgs to the left gastrocnemius muscle. The following gene therapy vectors were administered by intramuscular injection (IM) into the left gastrocnemius (GAS) muscle of 3 month old mdx/utrn^{+/-} mice, a DMD mouse model: scAAVrh.74.CMV.miR-29c alone, co-delivered with rAAVrh. 74.MCK.micro-dystrophin, and rAAVrh. 74.MCK.micro-dystrophin alone.

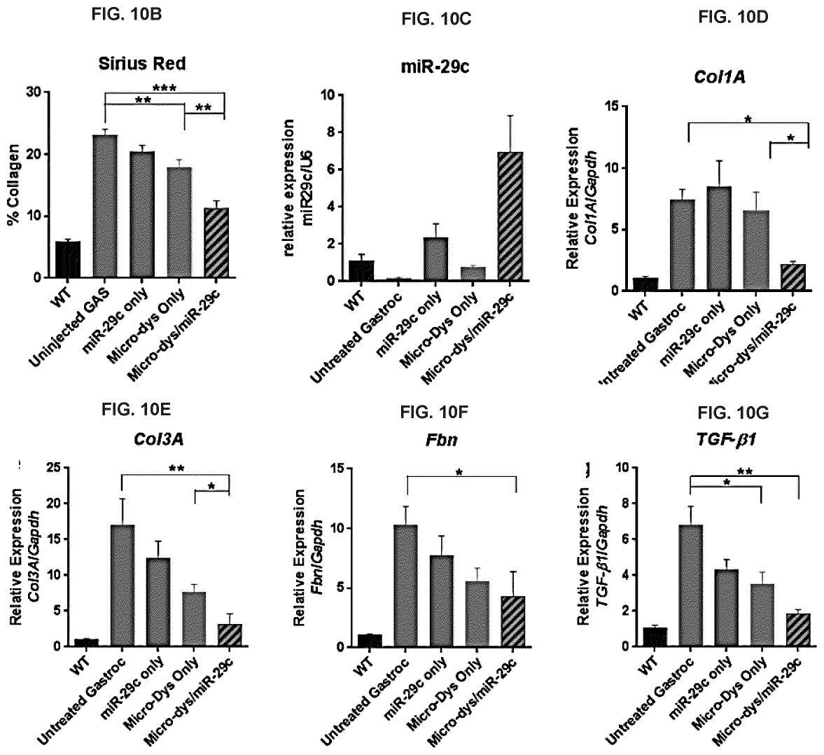

The pAAV.MCK.micro-dystrophin plasmid contains the human micro-dystrophin cDNA expression cassette flanked by AAV2 inverted terminal repeat sequences (ITR) as shown in FIG. 10. It is this sequence that was encapsidated into AAV rh.74 virions. The pAAV.MCK.micro-dystrophin plasmid was constructed by inserting the MCK expression cassette driving a codon optimized human micro-dystrophin cDNA sequence into the AAV cloning vector as described in Rodino-Klapac et al. (Mol Ther. 2010 January; 18(1): 109-17). A MCK promoter/enhancer sequence was used to drive muscle-specific gene expression and is composed of the mouse MCK core enhancer (206 bp) fused to the 351 bp MCK core promoter (proximal). After the core promoter, the 53 bp endogenous mouse MCK Exon1 (untranslated) is present for efficient transcription initiation, followed by the SV40 late 16S/19S splice signals (97 bp) and a small 5′UTR (61 bp). The intron and 5′ UTR are derived from plasmid pCMVß (Clontech). The micro-dystrophin cassette has a consensus Kozak immediately in front of the ATG start and a small 53 bp synthetic polyA signal for mRNA termination. The human micro-dystrophin cassette contains the (R4-R23/Δ71-78) domains. The complementary DNA was codon optimized for human usage and synthesized by GenScript (Piscataway, NJ).

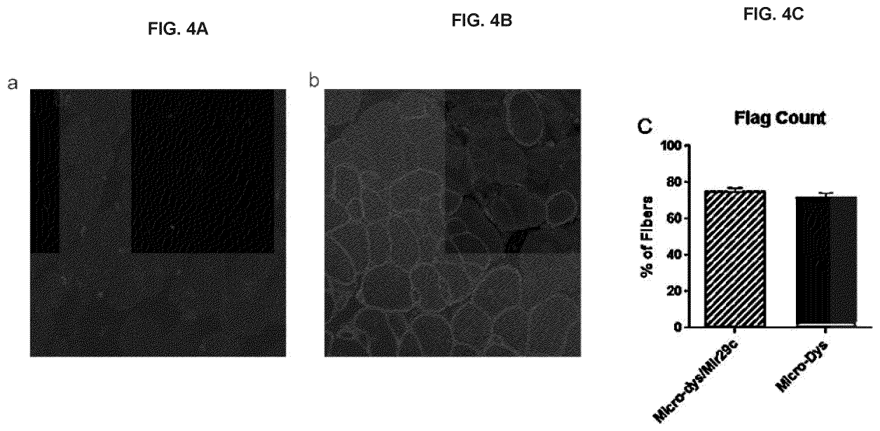

The mice were analyzed at 12 and 24 weeks post injection. First, the number of muscle fibers expressing micro-dystrophin was used to assess the efficacy of transgene delivery and to make sure we had similar levels of micro-dystrophin expressed in each group. We found that micro-dystrophin was not different between cohorts treated with micro-dystrophin alone (71.85±2.25%) compared with miR-29c/micro-dystrophin combination therapy (75.03±1.91%) (FIG. 4).

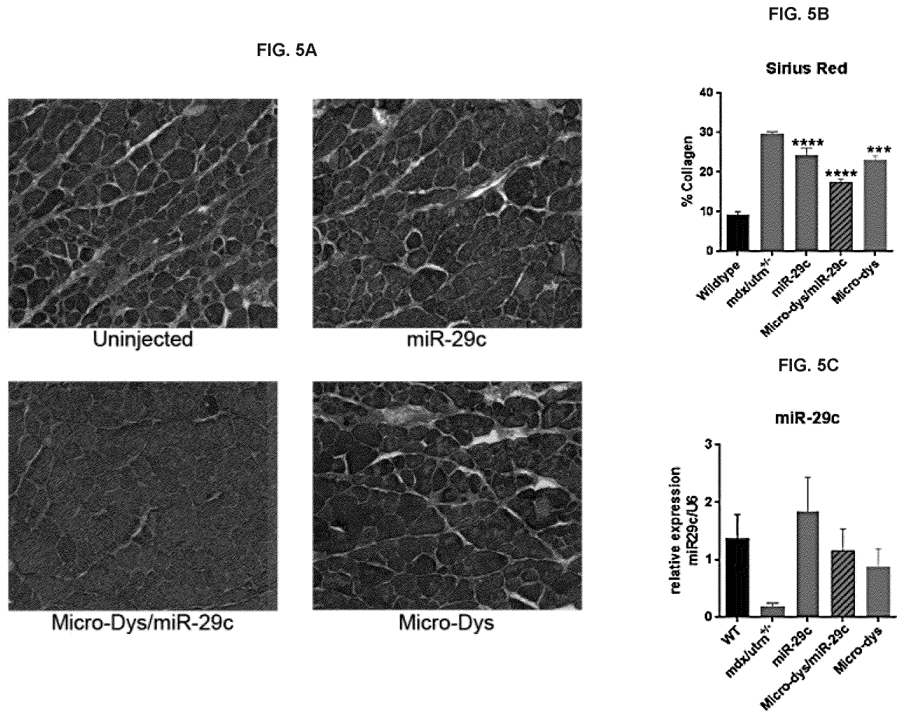

GAS muscle was analyzed 12 months post-injection to assess collagen accumulation by Sirius Red staining and subsequent quantification with ImageJ. Additional outcomes included miR-29c and collagen transcript levels, force measurements in the GAS muscle, fiber diameter measurements and western blot analysis for proteins involved in muscle regeneration (MyoD, Myogenin). The amount of fibrosis was analyzed by picrosirius red staining, which revealed a significant decrease in collagen staining throughout the GAS muscles in all treated groups (FIG. 5a) compared to the untreated contralateral mdx/utrn+/− GAS muscle or micro-dystrophin alone. Quantification of the picrosirius red staining shows that co-treated muscle had a 40.8% reduction in collagen compared to the untreated muscle (treated-17.47%±0.75 vs. untreated-29.5%±0.7) (FIG. 5b). To confirm expression of miR-29c, qRT-PCR was performed on the GAS muscle and all treatment groups had an increase in miR-29c compared to untreated muscle (FIG. 5c).

Analogous to DMD tissue, a significant reduction in miR-29c levels in mdx/utrn^{+/-} muscle was observed which correlated with increased fibrosis measured by picrosirius red staining. Following 3 months of treatment with scAAV.miR-29c alone, there was a significant reduction in fibrosis (treated-23.5%±1.3 vs. untreated-27.8%±0.6) in the GAS muscle. When co-delivered with micro-dystrophin, further reduction in collagen (41%) was observed by picrosirius red staining (combination treatment: 17.47%±0.75 vs. untreated: 29.5%±0.7) (p<0.0001) (FIG. 5b). To confirm expression of miR-29c, qRT-PCR was performed on the GAS muscle and all treatment groups had an increase in miR-29c compared to untreated muscle (FIG. 5b).

At 24 weeks post-injection, the results were similar to those observed 12 weeks post injection. There was a 47% reduction in collagen by picrosirius red staining compared to the untreated muscle (combination treatment: 16.5±1.23 vs. untreated: 31.07±0.93; p<0.0001) and a coincident increase in miR-29c transcript level.

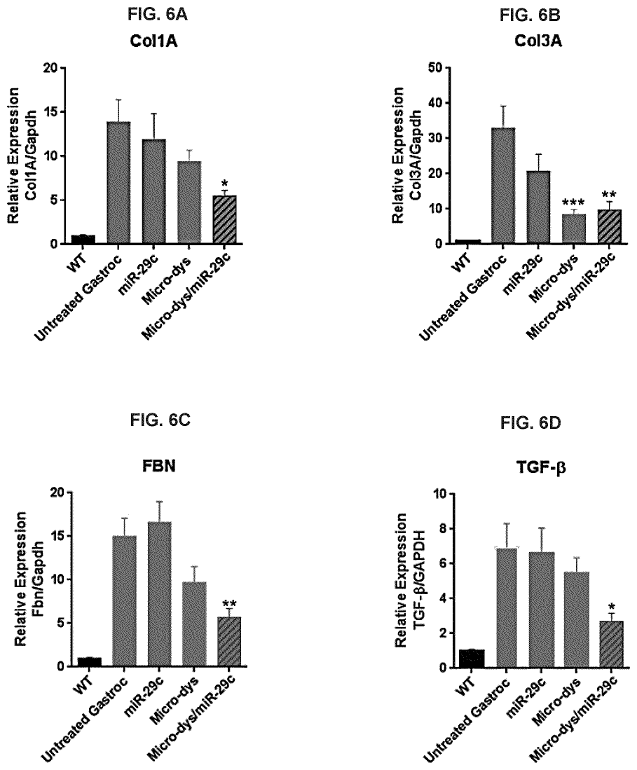

To further validate reduction of collagen observed by picrosirius red staining, qRT-PCR was performed on the muscle to quantify transcript levels of Col1A, Col3A and also another ECM component, fibronectin (Fbn). qRT-PCR analysis detected a decrease in Col1A and Col3A following each treatment, however only the cohort treated with both micro-dystrophin and miR-29c showed significant reduction (FIGS. 6a and 6b). The analysis revealed that Fbn was significantly reduced only in the co-treated cohort (FIG. 6c).

TGF-β1 has been previously shown to be up regulated in dystrophic muscle, likely playing a role in the initiation of the fibrotic cascade. TGF-β1 is a known pro-fibrotic cytokine that down regulates miR-29c and is responsible for conversion of myoblasts to myofibroblasts with an increase in collagen and muscle fibrogenesis. qRT-PCR analysis shows that co-treated muscle had significantly lower levels of TGF-β1 compared to uninjected muscle and either treatment alone (FIG. 6d). At 6 months post injection, co-treated muscle continued to show reduced Col1A, Col3A, Fbn and TGF-β1 levels, whereas only slight reductions in Col1A mRNA levels in the miR-29 and the micro-dystrophin only groups were observed

An increase in specific and absolute force was observed in the muscle treated with miR-29c alone compared to the untreated limb, which when combined with micro-dystrophin led to absolute and specific force that were not significantly different than wild-type. We also observed a significant increase in gastroc weight in those muscles that were co-treated.

Initial results using rAAV.miR-29c as an anti-fibrotic therapy suggest that there is beneficial effect with reduction in collagen levels, a key contributor in fibrosis. Moreover, when combined with micro-dystrophin to improve membrane stability, miR29 up regulation normalized muscle force.

### Example 6 Further Increase in Absolute Force and Added Protection from Contraction-induced Damage

Knowing that miR-29-treated muscle had a modest but significant increase in absolute and specific force, the combination therapy of miR-29c overexpression and micro-dystrophin gene replacement impact on muscle function was investigated. Twelve weeks post injection, we isolated the GAS for which we performed in vivo force measurements. The rAAVrh.74. MiR-29c vector described above in Example 2 and a rAAV

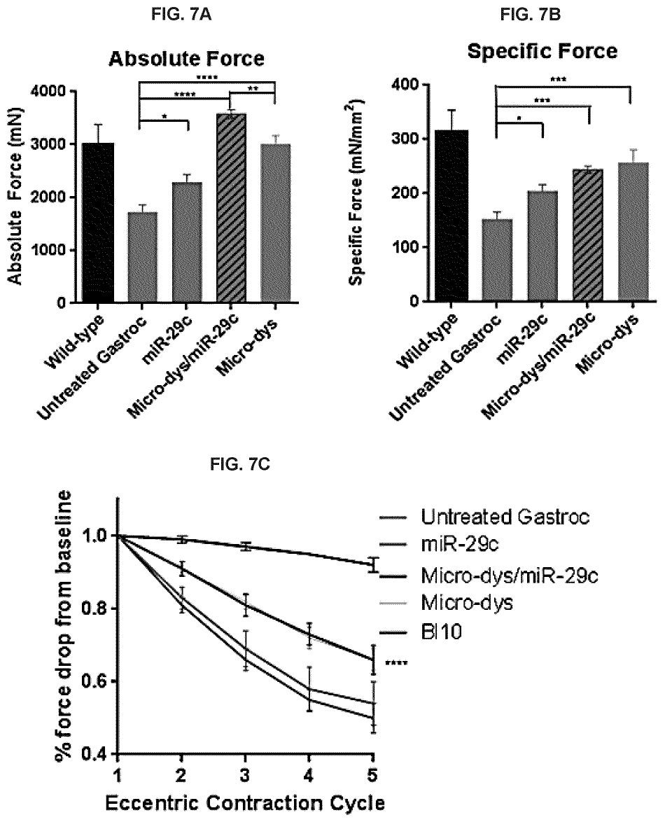

Co-treated rAAVrh.74.MiR-29c and rAAV expressing Micro-Dys treated GAS muscle showed significant improvement in absolute force when compared to untreated mdx/utrn^{+/-} GAS muscle (co-treated-3582.4±79.4 nM vs. mdx/utrn-untreated-1722±145.7 nM vs. wild-type-3005±167.3 nM) (FIG. 7), and also normalized specific force in rAAVrh.74.miR-29c/micro-dys treated GAS muscle specific improvement when compared to untreated GAS muscle (co-treated mice-244.2±6.6 nM/mm² vs. mdx/utrn-untreated-151.6±14.5 nM/mm² vs. 312.0±34.1 nM/mm²) (FIG. 7). Both absolute and specific force was not significantly different from wild-type controls.

Each GAS was subjected to a series of repeated eccentric contraction. By comparing the force ratio of each contraction versus the first contraction revealed that after the fifth contraction untreated muscle decayed to 0.54±0.06 versus co-treated 0.66±0.04 (p≤0.0001), which can be contributed to the micro-dystrophin since the micro-dystrophin alone also decayed to 0.66±0.04. The treated group was still significantly lower than wild-type that decayed to 0.92±0.02 (FIG. 7c). Similar findings were seen at 24 weeks post injection This data shows that reducing fibrosis and gene replacement leads to increase in both absolute and specific 5force and significantly protects muscle from contraction-induced injury.

### Example 7 Combination Treatment Increases Muscle Hypertrophy and Hyperplasia

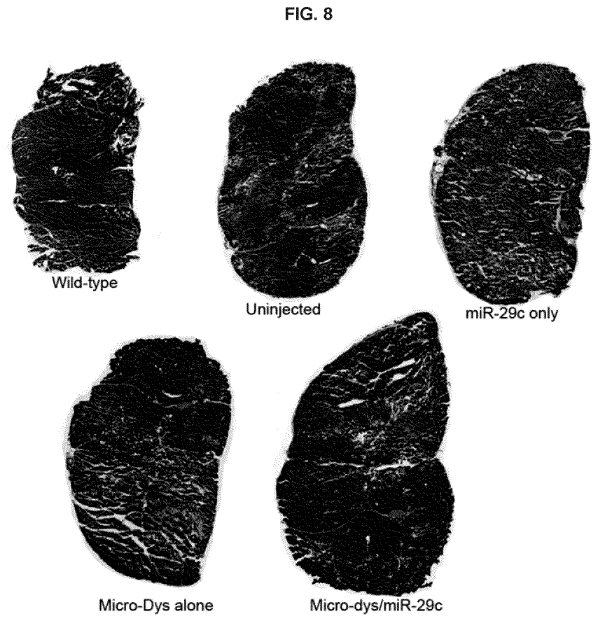

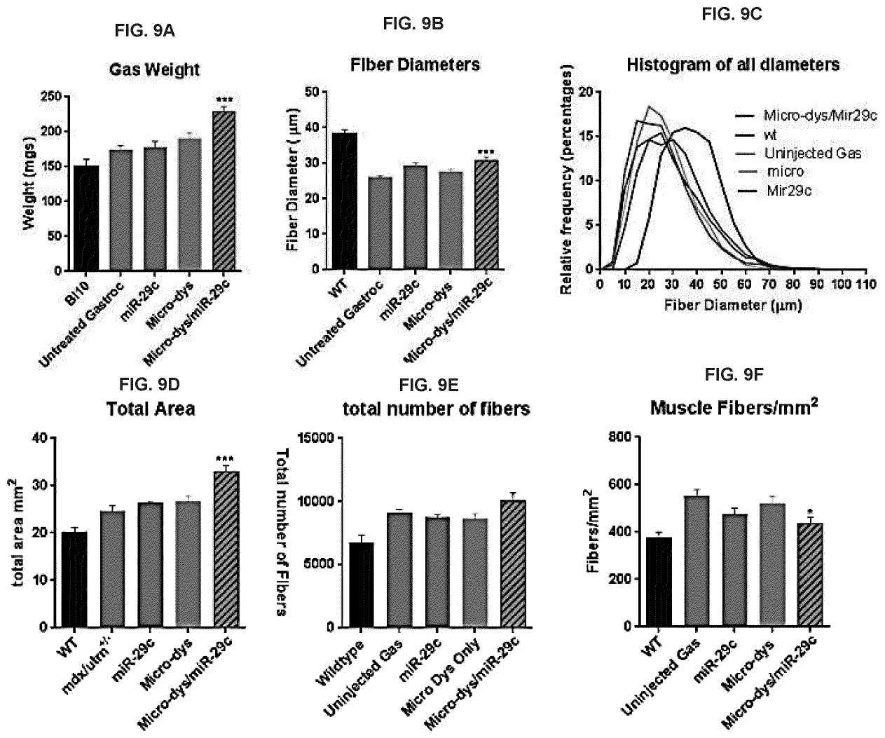

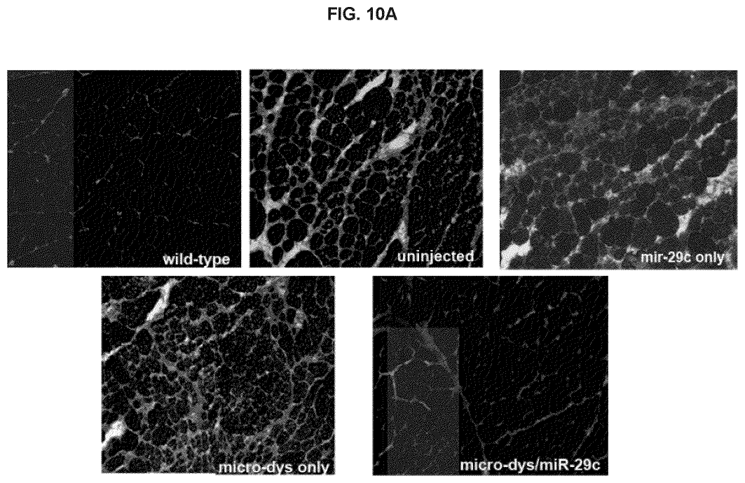

MIR-29c co-delivered with micro-dystrophin increased the overall weight of the injected gastroc compared to either one injected alone at three months of age (FIG. 8, FIG. 9a). To investigate the source of increased muscle mass, myofiber diameters are measured. miR-29c/μ-dys combination treatment demonstrated an increase in average fiber size. Comparing mdx/utrn^{+/-} controls with miR-29c/μ-dys treated mdx/utrn^{+/-}, the average diameter increased from 25.96 to 30.97 μm (FIG. 9b). The co-delivery produced a shift towards wild-type fiber size distribution (FIG. 9c). Although the average fiber size was increased does not explain the ˜30% increase in gross muscle weight. Total cross-sectional area of the muscle was also measured. Gastroc muscles from all groups were full slide scanned and the total area was measured. Muscles co-treated with micro-dys/miR-29c had a significant increase in cross sectional area compared to untreated and either treatment alone (uninjected: 24.6 vs. miR-29c: 26.3 vs. micro-dys: 26.6 vs. micro-dys/miR-29c: 33.1) (FIG. 8, FIG. 9d).

miR-29c has been reported it to play a role in the myoD/Pax7/myogenin pathway and it was hypothesized that miR-29c may be impacting regeneration and activation of satellite cells (muscle stem cells) to differentiate in myogenic lineage. To test this, the total number of muscle fibers from the full slide scanned images was counted. An increased number of muscle fibers following miR-29c/μ-dys combination treatment (FIG. 9e). Finally, given that muscle fiber diameters in mdx/utrn+/− mice are heterogeneous with many small fibers and some hypertrophic fibers, it was determined whether the number of fibers per unit area (cells/mm2) was affected with treatment. miR-29c/μ-dys combination treatment was not different than wild-type (FIG. 9f).

### Example 8 Early Treatment With Combination Prevents Fibrosis

In view of the potential importance of combinatorial miR-29c and micro-dystrophin as a prophylactic therapy for DMD, a cohort of younger mdx/utrn^{+/-} mice were treated at 4 weeks of age. Using the same paradigm as for other groups as described herein, the following treatments were compared for efficacy for prevention of fibrosis by intramuscular injection of GAS: scAA Vrh.74.CMV.miR-29c alone, ssAAVrh74.MCK.micro-dystrophin+scAA Vrh.74.CMV.miR-29c combination therapy, or ssAAVrh74.MCK.micro-dystrophin alone at the same dose. The mice were necropsied 12 weeks post injection. A significant decrease in collagen staining throughout the GAS muscles in all treated groups compared to the untreated contralateral mdx/utrn^{+/-} GAS muscle was observed (FIG. 10A). Quantification of the picrosirius red staining showed that muscle co-treated with micro-dystrophin/miR-29c had a 51% reduction in collagen compared to the untreated muscle (treated-11.32%±1.18 vs. untreated-23.15%±0.90) (p<0.0001) (FIG. 10) and qRT-PCR confirmed Col1A, Col3A, Fbn and TGF-β1 reduction following combinatorial therapy (FIG. 10D and E).

### Example 9 Early Combination Therapy Restores Force and Protects from Contraction-Induced Damage Better than Late Treatment

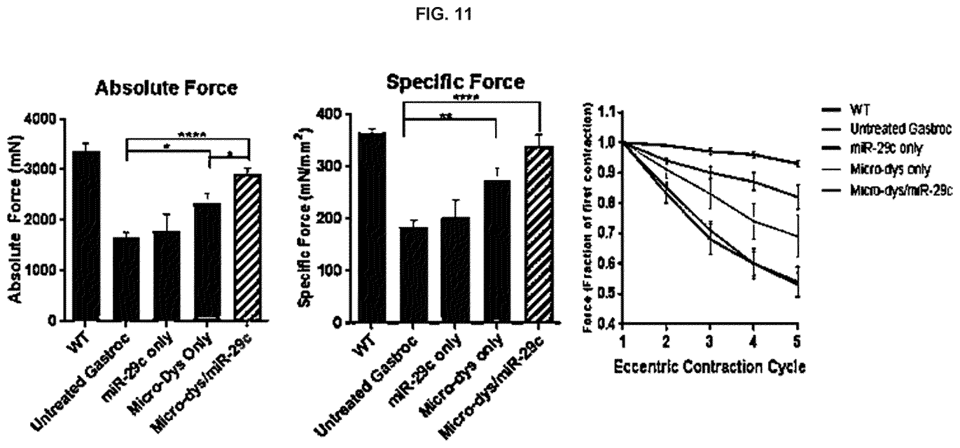

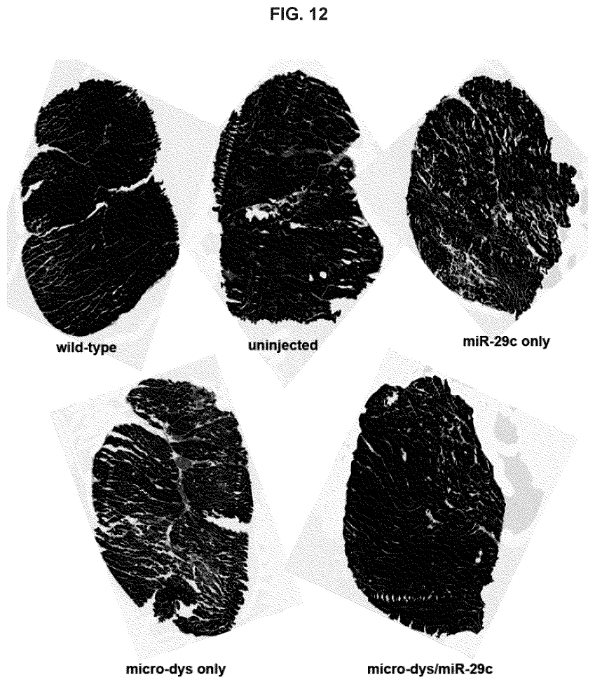

In vivo force measurement was carried out on the GAS of the mice treated early with the combination therapy as described in Example 8. In 4-week-old mdx/utrn^{+/-} mice, co-treatment using miR-29c/micro-dystrophin showed significant improvement in absolute force when compared to untreated mdx/utrn^{+/-} mice and there was no difference from wild type (co-treated: 2908±129.5 mN vs. untreated: 1639.4±116.9 mN vs. wild-type: 3369.73±154.1 mN). Specific force was also normalized to wild type levels following combinatorial therapy (co-treated 338.9±22.34 mN/mm2 vs. untreated 184.3±13.42 mN/mm² vs. WT 364.35±7.79 mN/mm²) (FIG. 11A and B and 12).

Next, each GAS was subjected to a series of repeat eccentric contractions. By comparing the force ratio of each contraction by the fifth contraction, untreated muscle decayed to 0.53±0.04 versus co-treated 0.82±0.04 (p≤0.0001). The combinatorial treatment group was slightly lower than wild type but not significantly different, which decayed to 0.93±0.01 (FIG. 11C). These data show that reducing fibrosis and gene replacement lead to increase in both absolute and specific force and significantly protects muscle from contraction-induced injury.

These experiments suggest that gene replacement should be started in the newborn period. Efforts are clearly moving in the direction of identifying DMD and other muscular dystrophies in the newborn period. The Ohio Newborn Screening Study illustrates the potential for identification of DMD in newborns using CK 7 Neurol. as a biomarker (>2000 U/L) with DNA confirmation on the same dried blood spot (Mendell et al., Ann. Neurol. 71:304-313, 2012). This methodology is now being extended to other states in the USA (PPMD May 16, 2016: Next Steps with Newborn Screening) and in other countries, particularly the UK (UK National Screening Committee) and China (Perkin Elmer™ launches screening in China).

miR-29 has also shown promise as a treatment modality for cardiac, pulmonary, and liver fibrosis. Myocardial infarction in mice and humans is associated with miR-29 down-regulation. Rooij et al. (Proc. Natl. Acad. Sci, USA 105:13027-13032, 2008) demonstrated that exposing fibroblasts to a miR-29b mimic decreased collagen transcripts providing a path for clinical translation for cardiac fibrosis. Subsequent studies showed that in a bleomycin-induced pulmonary fibrosis mouse model, attenuation of fibrosis could be achieved using the Sleeping Beauty (SB) transposon system-based delivery of miR-29b.14. Currently, a miR-29b mimic is in a clinical Phase 1 Safety-Tolerability local intradermal trial in healthy volunteers (miRagen Therapeutics™ MRG-201). Compared to miR-29 oligonucleotide delivery that would require repeated administration related to the half-life of the oligonucleotides, AAV gene therapy could potentially provide a path for single-delivery gene transfer.

### Example 10 Treatment with Muscle Specific Expression of miR-29 and Micro-dystrophin Reduced Fibrosis and ECM Expression

AAV vectors comprising the miR29c sequence and a muscle specific promoter MCK were also generated and tested as a combination therapy with AAV vectors expressing micro-dystrophin. To generate the rAAV vector, referred to herein as rAAV.MCK.miR29c, the 22 nucleotide miR29c sequence (target strand SEQ ID NO: 3 and guide strand SEQ ID NO: 4) was cloned into a miR-30 scaffold driven by a MCK promoter (SEQ ID NO: 11). The expression cassette (SEQ ID NO: 12) was cloned into a single stranded AAV plasmid and packaged using AAVrh74, a serotype known to express well in muscle. The miR-29c cDNA was synthesized using a custom primer containing the miR-29c target (sense) strand, miR-30 stem loop and miR-29c guide (antisense) strand in the miR-30 backbone. Three bases of the miR-29c sequence were modified. This sequence was then cloned into a single stranded AAV ITR containing plasmid driven by the MCK promoter and poly A sequence.

The pAAV.MCK.miR29C plasmid contains the mir29c cDNA in a miR-30 stem loop backbone flanked by AAV2 inverted terminal repeat sequences (ITR). It is this sequence that was encapsidated into AAVrh74 virions. In addition, a few nucleotides with in the miR-29c target sequence were changed to mimic Watson-crick pairing at this site as in shRNA-miR(luc). According to ShRNA-luc design, the hairpin should be perfectly complementary throughout its length. Plus, the more changes to the passenger strand, the more likely the elimination of any endogenous mechanism that regulates miR-29 processing that could recognize the miRNA via the stem. The 19^{th} base of the guide strand was modified to a cytosine to mimic the nucleotide that precedes the cleavage site in natural mi-29c sequence and the corresponding base on the other strand was changed to preserve pairing.

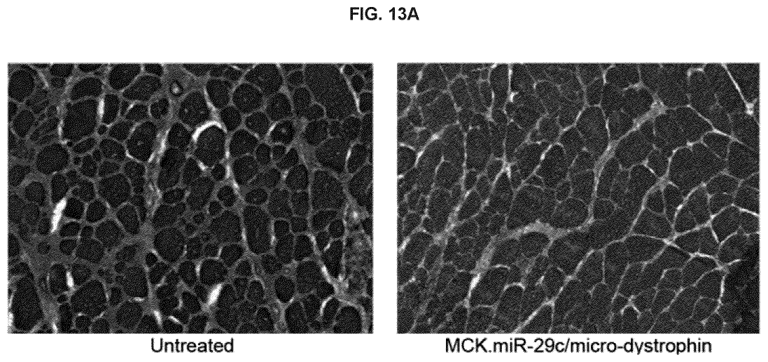

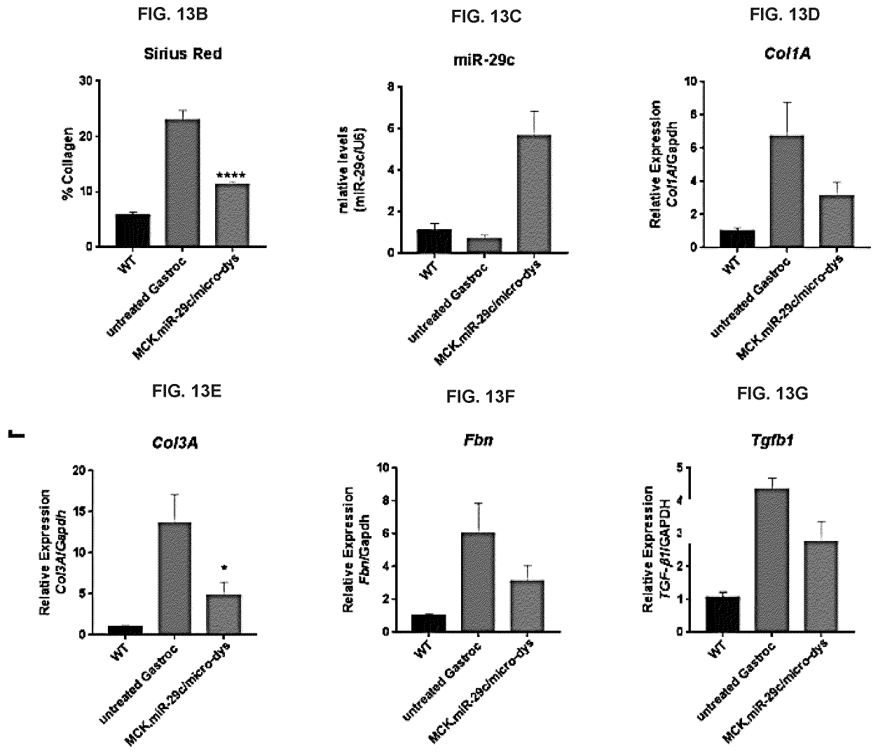

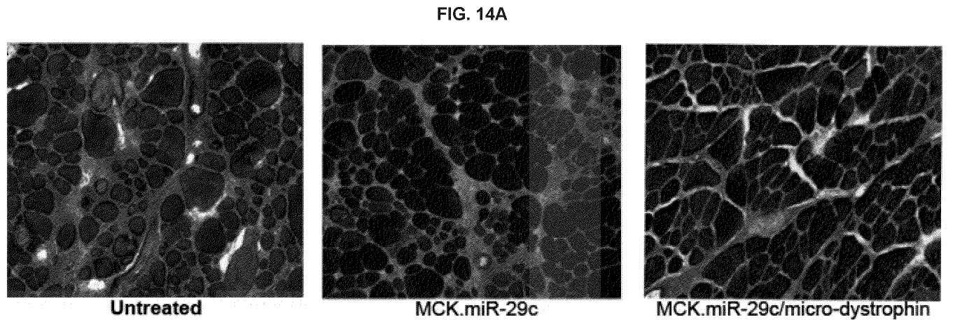

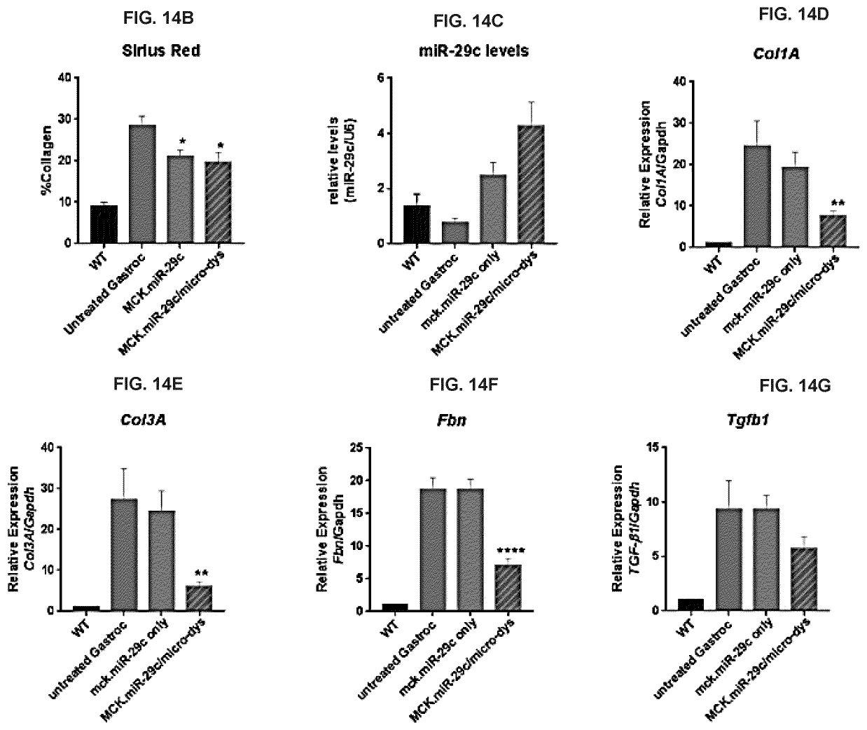

Early treatment of AAV.MCK.miR-29c/micro-dystrophin combination therapy was more effective at reducing fibrosis and ECM expression. 4-5-week-old mdx/utrn^{+/-} mice received an intramuscular injection of rAAVrh.74.MCK.MIR-29c and rAAVrh74.MCK.micro-dystrophin at 5x1011 vgs to the left gastrocnemius muscle as described in Example 5. The muscles were harvested twelve weeks post injection. Picrosirius red staining of muscle harvested from uninjected and mice injected with combination therapy of rAAV.MCK.miR-29c/rAAV.MCK.micro-dystrophin showed co-treated muscle had a 50.9% reduction in collagen compared to untreated GAS muscle (See FIGS. 13a and 13b). qRT-PCR confirmed an increase in miR-29c transcript levels in the treated cohort (FIG. 13c). Semi-quantitative qRT-PCR showed a significant reduction in Collagen A1 and Collagen 3A (FIG. 13d, e), Fibronectin (FIG. 13f) and Tgfβ1 (FIG. 13g) levels in the AAV.MCK.miR-29c/AAV.micro-dystrophin treated muscle compared to the contralateral limb therapies. (*p<0.05, ****p<0.0001).Late treatment of AAV.MCK.miR-29c/micro-dystrophin combination therapy is effective at reducing fibrosis and ECM expression. Three month old mdx/utrn^{+/-} mice received an intramuscular injection of rAAVrh.74.MCK.MiR-29c and rAAVrh.74.MCK.micro-dystrophin at 5×10¹¹ vgs to the left gastrocnemius muscle as described in Example 5. The muscles were harvested twelve weeks post injection. Picrosirius red staining of untreated, AAV.MCK.miR-29c and AAV.MCK.miR-29c/AAV.micro-dystrophin treated muscle showed co-treated muscle had a 30.3% reduction in collagen compared to untreated GAS muscle (See FIGS. 14a and 14b) qRT-PCR confirmed an increase in miR-29c transcript levels in the treated cohorts (FIG. 14c). Semi-quantitative qRT-PCR shows a significant reduction in Collagen 1A and Collagen 3A (FIG. 14d, e), Fibronectin (FIG. 14f) and Tgfβ1 (FIG. 14G) levels in the AAV.miR-29c/AAV.micro-dystrophin treated muscle compared to the contralateral limb. One-way ANOVA. All data represent mean±SEM. (**p<0.01, ****p<0.0001).

### Example 11 Early Combination Therapy Restores Force and Protects from Contraction-Induced Damage Better than Late Treatment

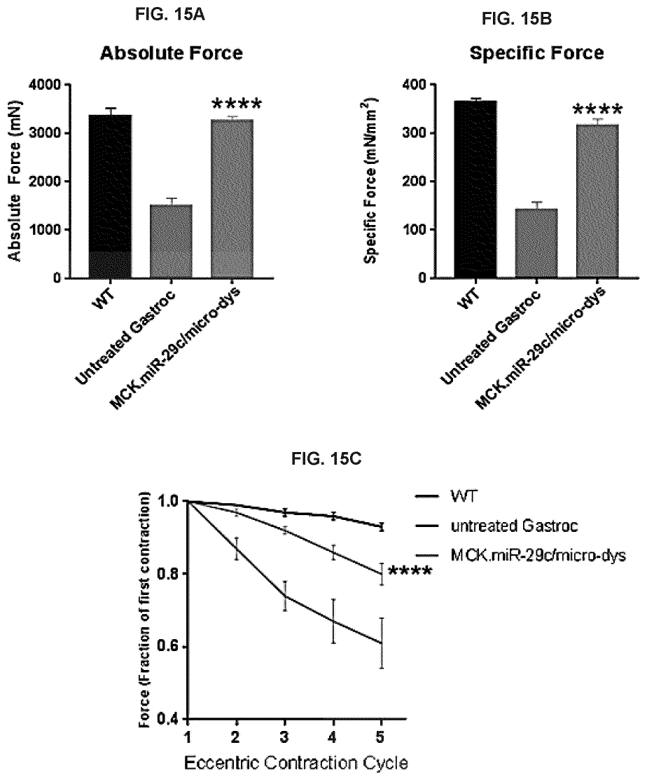

In vivo force measurement was carried out on the GAS of the mice treated early with the muscle-specific expression of miR-29 and micro-dystrophin. as described in Examples 8 and 9. In 4-week-old mdx/utrn^{+/-} mice, co-treatment using rAAV.MCK.miR-29c/and rAAV expressing micro-dystrophin showed significant improvement in absolute force when compared to untreated mdx/utrn^{+/-} mice and there was no difference from wild type (FIG. 15a). Specific force was also normalized to wild type levels following combination therapy (FIG. 15b).

Muscles were then assessed for loss of force following repetitive eccentric contractions as described in Example 9. Mice co-treated with rAAV.MCK.miR-29c/rAAV.MCK.micro-dystrophin and rAAV.MCK.micro-dystrophin alone showed a protection from loss of force compared with untreated mdx/utrn^{+/-} muscles (FIG. 15c).

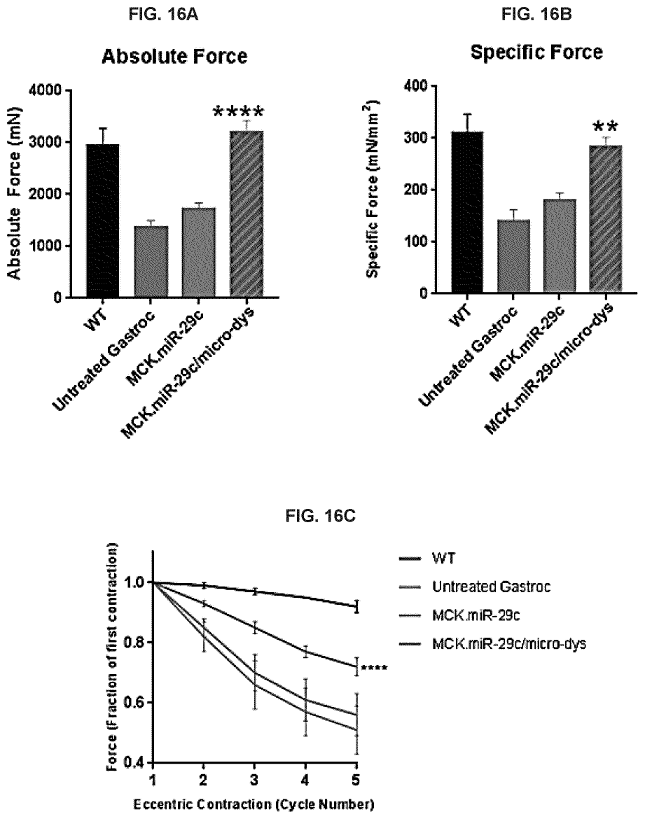

In 12-week-old mdx/utrn^{+/-} mice, co-treatment using rAAV.MCK.miR-29c/and rAAV expressing micro-dystrophin restored force and protected against contraction-induced damage. Measurement of absolute (FIG. 16a) and normalized specific force (FIG. 16b) following tetanic contraction rAAV.MCK.miR-29c and rAAV expressing micro-dystrophin injected GAS muscles were significantly increased compared to untreated mdx/utrn^{+/-} muscle. Subsequently, muscles were assessed for loss of force following repetitive eccentric contractions as described in Example 9. Mice co-treated with MCK.miR-29c/micro-dystrophin showed a protection from loss of force compared with untreated mdx/utrn^{+/-} muscles (FIG. 16c). These data show that reducing fibrosis and gene replacement lead to increase in both absolute and specific force and significantly protects muscle from contraction-induced injury.

### Example 12 Early Combination Treatment Increases Muscle Hypertrophy and Hyperplasia

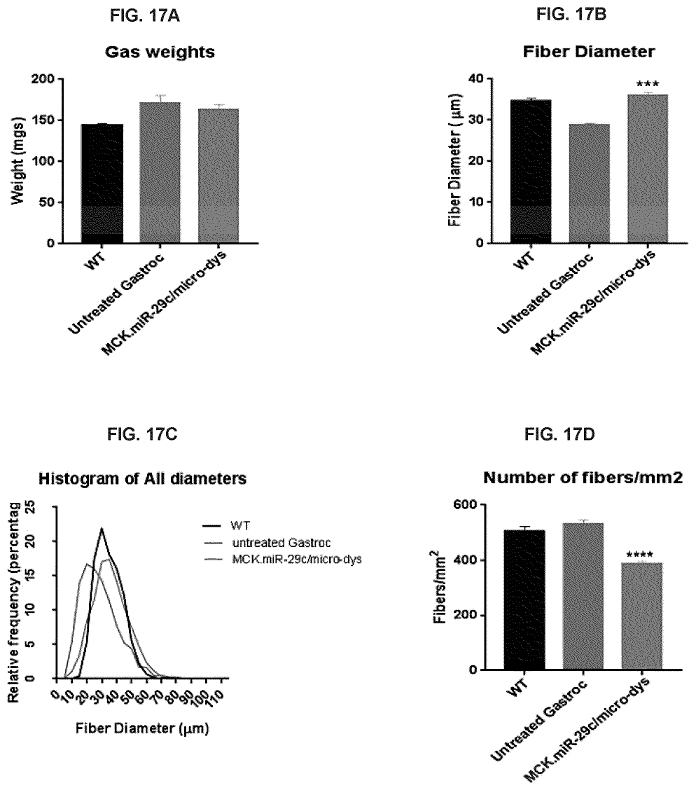

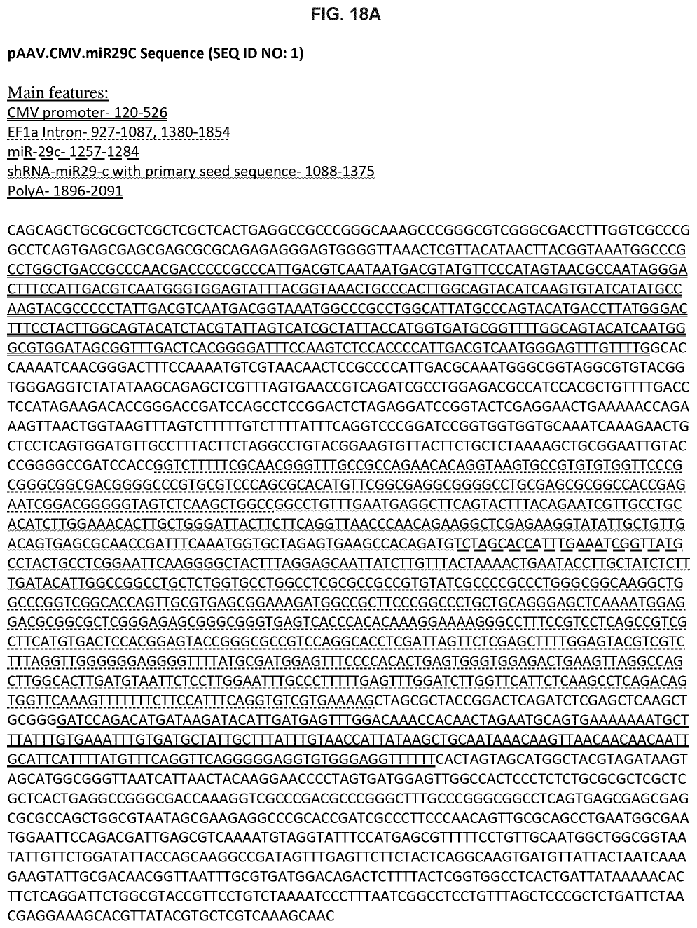

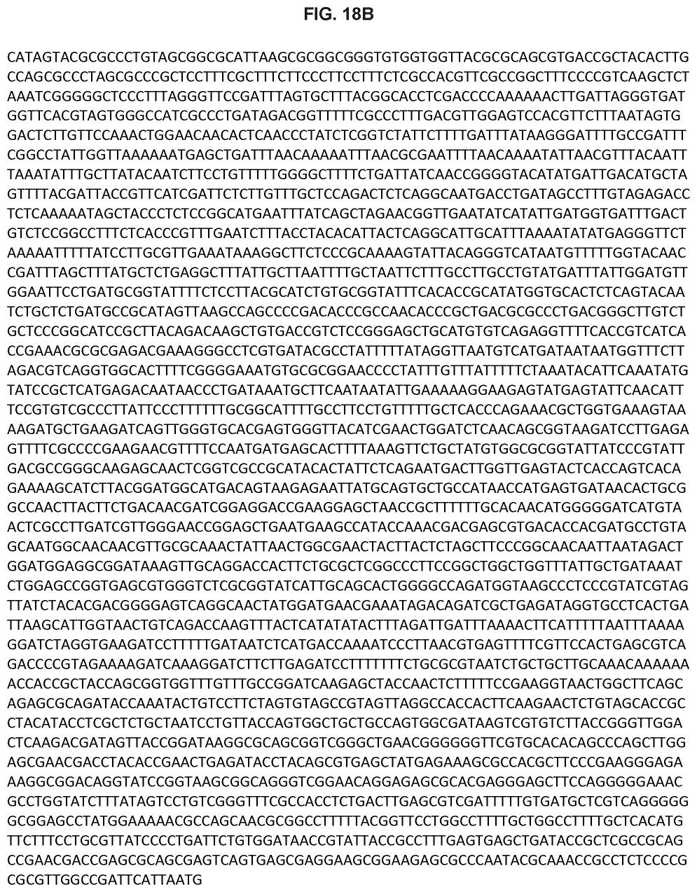

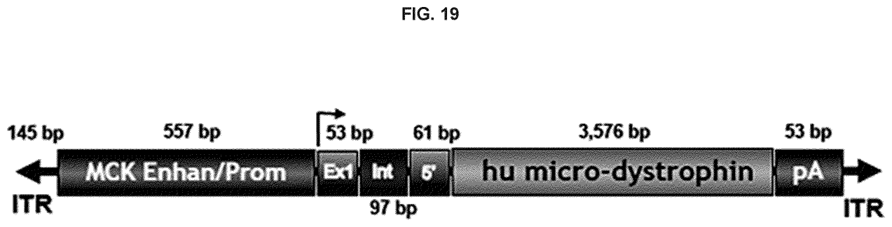

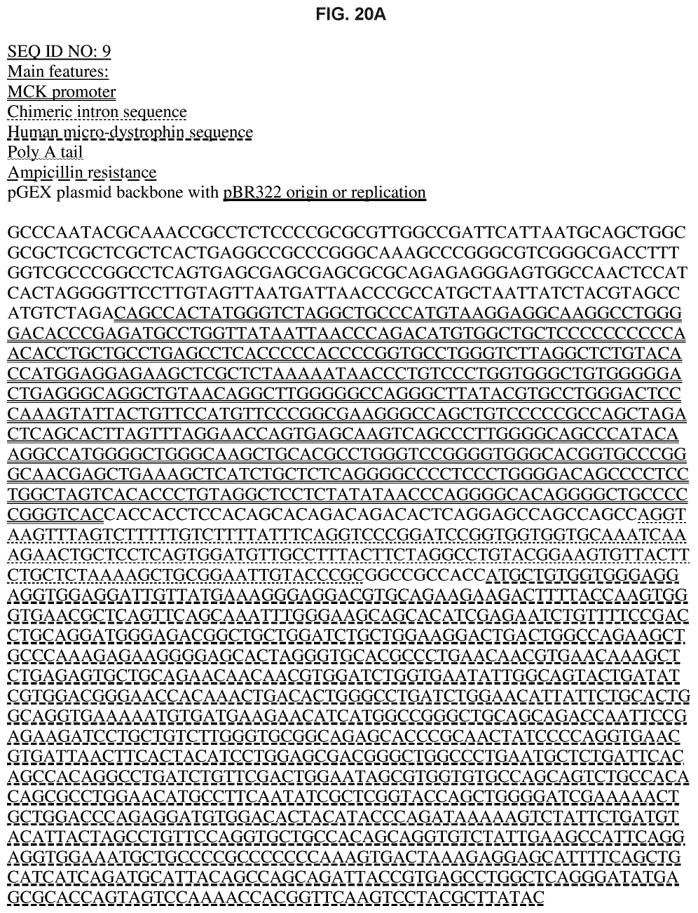

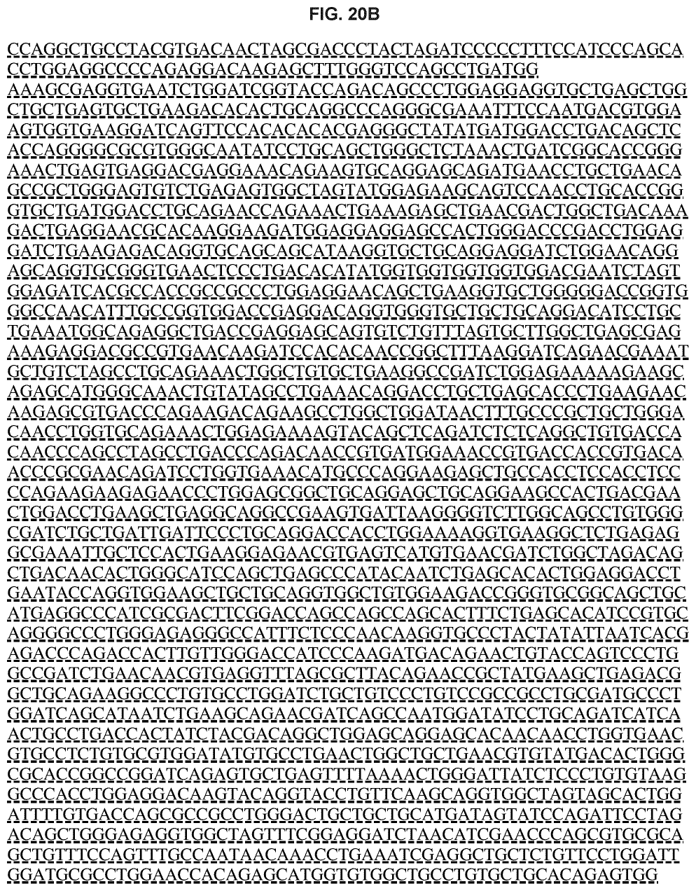

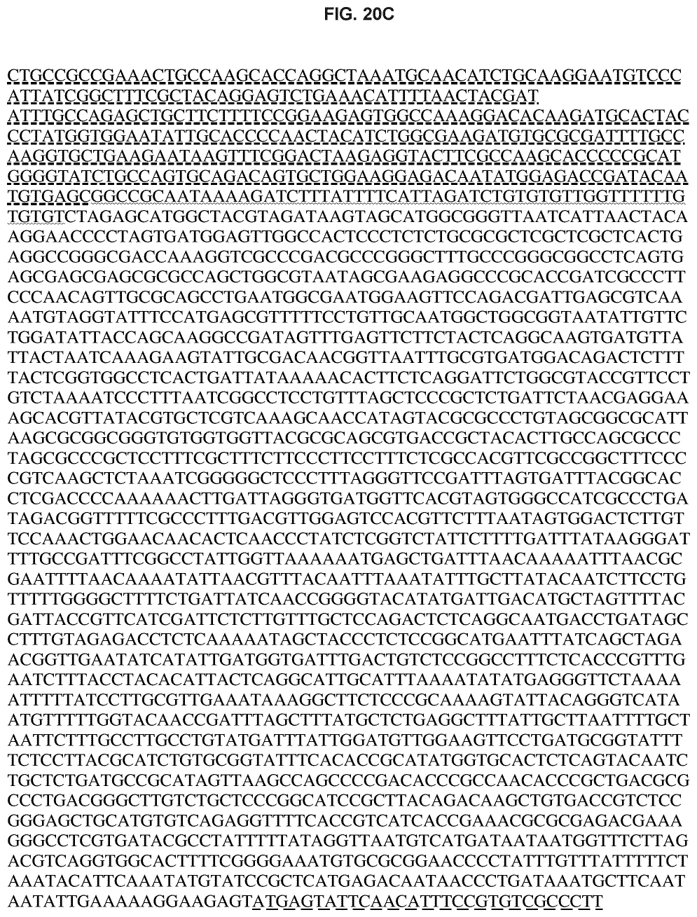

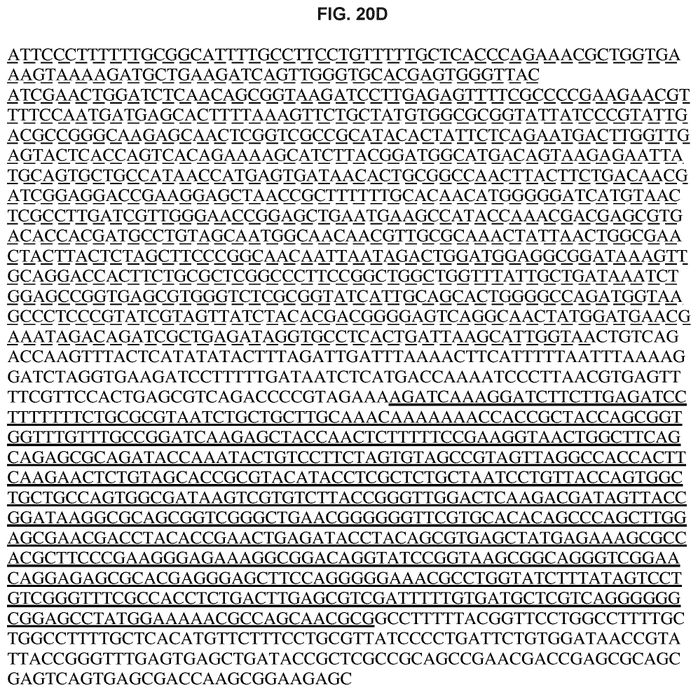

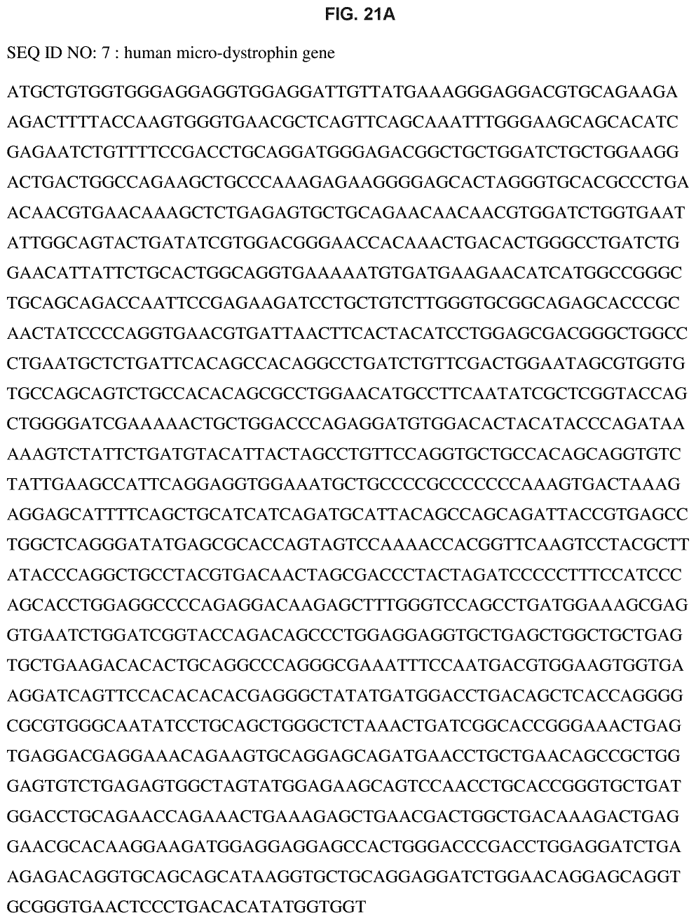

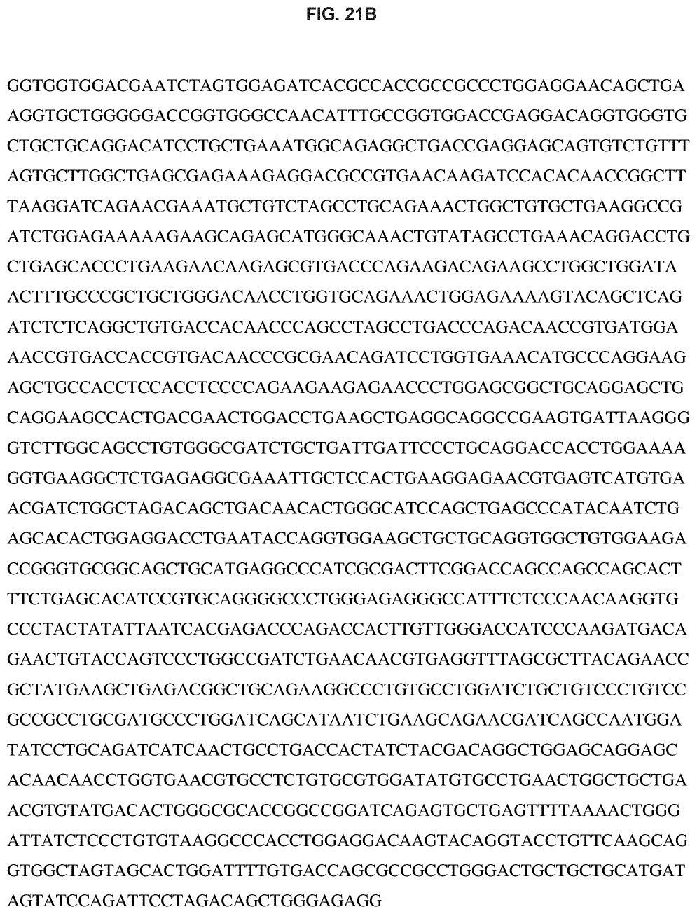

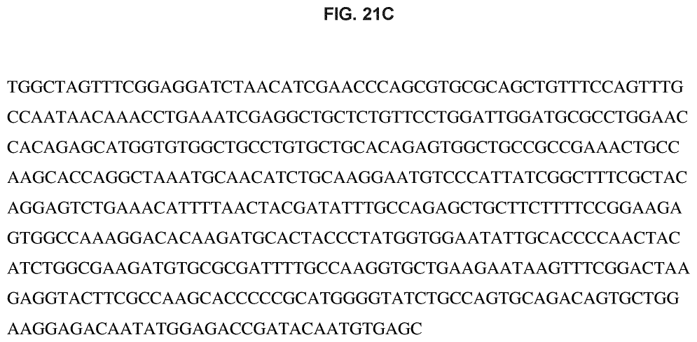

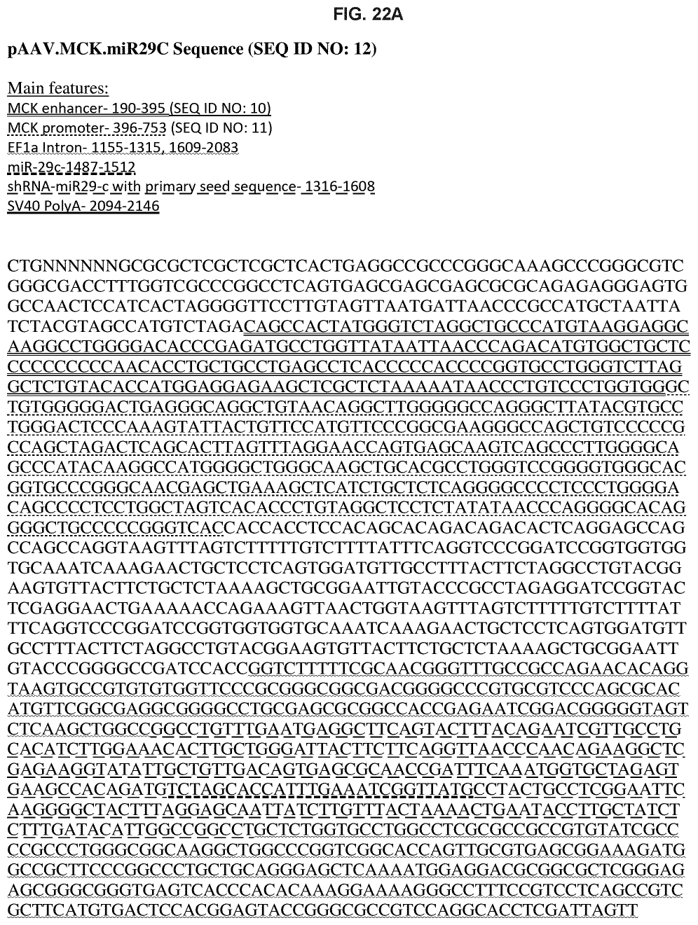

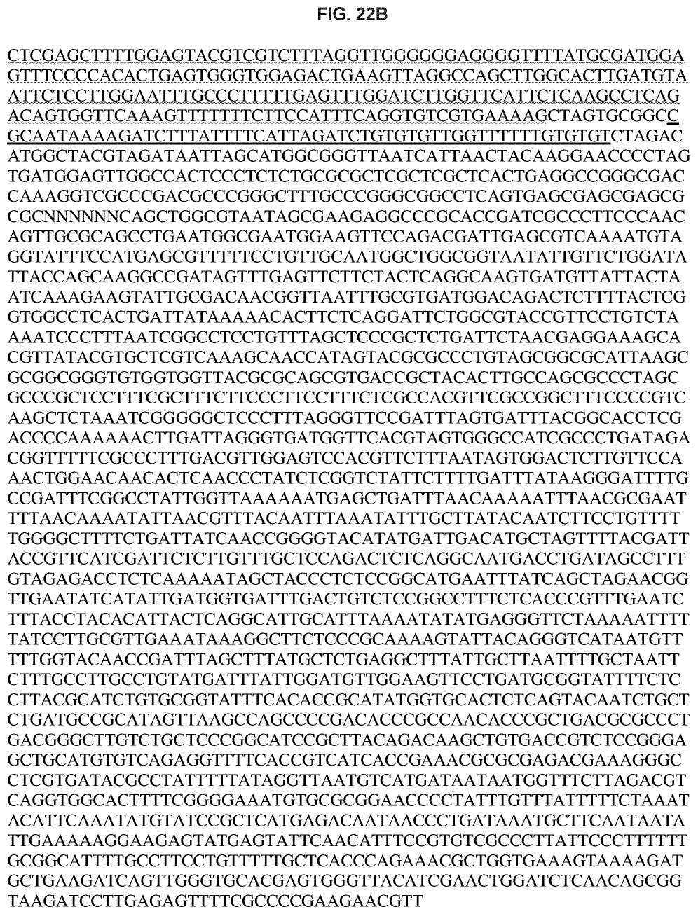

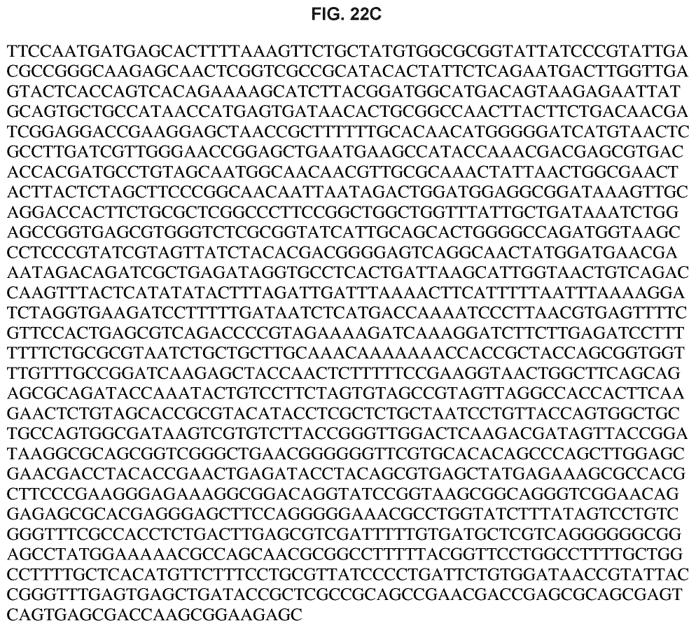

Co-delivery of rAAV.MCK.miR-29 with rAAV expressing micro-dystrophin did not increase overall weight of the injected gastroc compared to either one injected alone at three months post-injection (FIG. 17a). Myofiber diameters were also measured. miR-29c/micro-dystrophin combination treatment demonstrated an increase in average fiber size. Comparing mdx/utrn^{+/-} controls with miR-29c/micro-dystrophin treated mdx/utrn^{+/-}, the average diameter increased from 28.96 to 36.03 μm (FIG. 17b). The co-delivery produced a shift towards wild-type fiber size distribution (FIG. 17c). The number of muscle fibers per mm² in the miR-29c/micro-dystrophin combination treatment was significantly less than untreated mice and wild-type (FIG. 17d; ***p<0.01, ****p<0.0001).

### REFERENCES

1. Hoffman, E. P., Brown, R. H., Jr. & Kunkel, L. M. Dystrophin: the protein product of the Duchenne muscular dystrophy locus. Cell 51, 919-928 (1987).
2. Straub, V. & Campbell, K. P. Muscular dystrophies and the dystrophin-glycoprotein complex. Curr Opin Neurol 10, 168-175 (1997).
3. Sacco, A., et al. Short telomeres and stem cell exhaustion model Duchenne muscular dystrophy in mdx/mTR mice. Cell 143, 1059-1071 (2010).
4. Wallace, G. Q. & McNally, E. M. Mechanisms of muscle degeneration, regeneration, and repair in the muscular dystrophies. Annu Rev Physiol 71, 37-57 (2009).
5. Zhou, L. & Lu, H. Targeting fibrosis in Duchenne muscular dystrophy. J Neuropathol Exp Neurol 69, 771-776 (2010).
6. Desguerre, I., et al. Endomysial fibrosis in Duchenne muscular dystrophy: a marker of poor outcome associated with macrophage alternative activation. J Neuropathol Exp Neurol 68, 762-773 (2009).
7. Kim, J., et al. microRNA-directed cleavage of ATHB15 mRNA regulates vascular development in Arabidopsis inflorescence stems. Plant J 42, 84-94 (2005).
8. Ambros, V. MicroRNA pathways in flies and worms: growth, death, fat, stress, and timing. Cell 113, 673-676 (2003).
9. Eisenberg, I., et al. Distinctive patterns of microRNA expression in primary muscular disorders. Proc Natl Acad Sci U S A 104, 17016-17021 (2007).
10. Jiang, X., Tsitsiou, E., Herrick, S. E. & Lindsay, M. A. MicroRNAs and the regulation of fibrosis. FEBS J 277, 2015-2021 (2010).
11. van Rooij, E., et al. Dysregulation of microRNAs after myocardial infarction reveals a role of miR-29 in cardiac fibrosis. Proc Natl Acad Sci U S A 105, 13027-13032(2008).
12. Cacchiarelli, D., et al. MicroRNAs involved in molecular circuitries relevant for the Duchenne muscular dystrophy pathogenesis are controlled by the dystrophin/nNOS pathway. Cell Metab 12, 341-351 (2010).
13. DiPrimio, N., McPhee, S. W. & Samulski, R. J. Adeno-associated virus for the treatment of muscle diseases: toward clinical trials. Curr Opin Mol Ther 12, 553-56 0(2010).
14. Mendell, J. R., et al. Sustained alpha-sarcoglycan gene expression after gene transfer in limb-girdle muscular dystrophy, type 2D. Ann Neurol 68, 629-638 (2010).
15. Mendell, J. R., et al. Limb-girdle muscular dystrophy type 2D gene therapy restores alpha-sarcoglycan and associated proteins. Ann Neurol 66, 290-297 (2009).
16. Mendell, J. R., et al. A phase 1/2a follistatin gene therapy trial for becker muscular dystrophy. Molecular therapy: the journal of the American Society of Gene Therapy 23, 192-201 (2015).
17. Carnwath, J. W. & Shotton, D. M. Muscular dystrophy in the mdx mouse: histopathology of the soleus and extensor digitorum longus muscles. J Neurol Sci 80, 39-54 (1987).
18. Coulton, G. R., Morgan, J. E., Partridge, T. A. & Sloper, J. C. The mdx mouse skeletal muscle myopathy: I. A histological, morphometric and biochemical investigation. Neuropathol Appl Neurobiol 14, 53-70 (1988).
19. Cullen, M. J. & Jaros, E. Ultrastructure of the skeletal muscle in the X chromosome-linked dystrophic (mdx) mouse. Comparison with Duchenne muscular dystrophy. Acta Neuropathol 77, 69-81 (1988).
20. Dupont-Versteegden, E. E. & McCarter, R. J. Differential expression of muscular dystrophy in diaphragm versus hindlimb muscles of mdx mice. Muscle Nerve 15, 1105-1110 (1992).
21. Stedman, H. H., et al. The mdx mouse diaphragm reproduces the degenerative changes of Duchenne muscular dystrophy. Nature 352, 536-539 (1991).
22. Deconinck, A. E., et al. Utrophin-dystrophin-deficient mice as a model for Duchenne muscular dystrophy. Cell 90, 717-727 (1997).
23. Grady, R. M., et al. Skeletal and cardiac myopathies in mice lacking utrophin and dystrophin: a model for Duchenne muscular dystrophy. Cell 90, 729-738 (1997).
24. Love, D. R., et al. An autosomal transcript in skeletal muscle with homology to dystrophin. Nature 339, 55-58 (1989).
25. Tinsley, J. M., et al. Primary structure of dystrophin-related protein. Nature 360, 591-593 (1992).
26. Tinsley, J., et al. Expression of full-length utrophin prevents muscular dystrophy in mdx mice. Nat Med 4, 1441-1444 (1998).
27. Squire, S., et al. Prevention of pathology in mdx mice by expression of utrophin: analysis using an inducible transgenic expression system. Hum Mol Genet 11, 3333-3344 (2002).
28. Rafael, J. A., Tinsley, J. M., Potter, A. C., Deconinck, A. E. & Davies, K. E. Skeletal muscle-specific expression of a utrophin transgene rescues utrophin-dystrophin deficient mice. Nat Genet 19, 79-82 (1998).
29. Zhou, L., et al. Haploinsufficiency of utrophin gene worsens skeletal muscle inflammation and fibrosis in mdx mice. J Neurol Sci 264, 106-111 (2008).
30. Gutpell, K. M., Hrinivich, W. T. & Hoffman, L. M. Skeletal Muscle Fibrosis in the mdx/utrn+/− Mouse Validates Its Suitability as a Murine Model of Duchenne Muscular Dystrophy. PloS one 10, e0117306 (2015).
31. Rodino-Klapac, L. R., et al. Micro-dystrophin and follistatin co-delivery restores muscle function in aged DMD model. Human molecular genetics 22, 4929-4937 (2013).
32. Cushing, L., et al. MIR-29 is a Major Regulator of Genes Associated with Pulmonary Fibrosis. Am J Respir Cell Mol Biol (2010).
33. Roderburg, C., et al. Micro-RNA profiling reveals a role for miR-29 in human and murine liver fibrosis. Hepatology 53, 209-218 (2011).
34. Nevo, Y., et al. The Ras antagonist, farnesylthiosalicylic acid (FTS), decreases fibrosis and improves muscle strength in dy/dy mouse model of muscular dystrophy. PloS one 6, e18049 (2011).
35. Rodino-Klapac, L. R., et al. A translational approach for limb vascular delivery of the micro-dystrophin gene without high volume or high pressure for treatment of Duchenne muscular dystrophy. J Transl Med 5, 45 (2007).
36. Mulieri, L. A., Hasenfuss, G., Ittleman, F., Blanchard, E. M. & Alpert, N. R. Protection of human left ventricular myocardium from cutting injury with 2,3-butanedione monoxime. Circ Res 65, 1441-1449 (1989).
37. Rodino-Klapac, L. R., et al. Persistent expression of FLAG-tagged micro dystrophin in nonhuman primates following intramuscular and vascular delivery. Molecular therapy: the journal of the American Society of Gene Therapy 18, 109-117 (2010).
38. Grose, W. E., et al. Homologous recombination mediates functional recovery of dysferlin deficiency following AAV5 gene transfer. PloS one 7, e39233 (2012).
39. Liu, M., et al. Adeno-associated virus-mediated microdystrophin expression protects young mdx muscle from contraction-induced injury. Mol Ther 11, 245-256 (2005).

What is claimed is:

1. A recombinant adeno-associated virus (rAAV) vector comprising a-micro-dystrophin gene comprising a nucleotide sequence encoding the amino acid sequence set forth in SEQ ID NO: 8.

2. The rAAV vector of claim 1, further comprising a selectable marker.

3. The rAAV vector of claim 1, wherein the rAAV vector has an AAV serotype selected from AAV-1, AAV-2, AAV-3, AAV-4, AAV-5, AAV-6, AAV-7, AAV-8, AAV-9, AAV-10, AAV-11, AAV-12, AAV-13, or AAV rh.74, or a variant of each thereof.

4. The rAAV vector of claim 3, wherein the AAV serotype is of serotype AAVrh.74, or a variant thereof.

5. The rAAV vector of claim 1, wherein the nucleotide sequence further comprises in the 5′ to 3′ direction an inverted terminal repeat (ITR), a muscle-specific control element, a chimeric intron sequence, the micro-dystrophin gene, a poly A tail, and an ITR.

6. The rAAV vector of claim 5, wherein the muscle-specific control element comprises the nucleotide sequence set forth in SEQ ID NO: 10 or SEQ ID NO: 11.

7. The rAAV vector of claim 5, wherein the chimeric intron sequence comprises nucleotides 844-993 of SEQ ID NO:9.

8. The rAAV vector of claim 5, wherein the poly A tail comprises nucleotides 4585 to 4640 of SEQ ID NO:9.

9. A composition comprising the rAAV vector of claim 1, and a pharmaceutically acceptable carrier.

10. A method for one or more of: treating muscular dystrophy; reducing or preventing fibrosis; increasing muscular force; or increasing muscle mass, each in a subject in need thereof comprising administering to the subject the composition of claim 9.

11. The method of claim 10, wherein the composition is administered before fibrosis is observed in the subject or before muscle force is reduced in the subject, or before muscle mass is reduced in the subject.

12. The method of claim 10, wherein the subject is suffering from Duchenne muscular dystrophy.

13. The method of claim 10, wherein the composition is administered by intramuscular administration, intravenous injection, parenteral administration or systemic administration.

14. A method of producing a functional micro-dystrophin protein comprising infecting a host cell with the rAAV vector of claim 1 and expressing a functional micro-dystrophin protein in the host cell.

15. A method of producing a functional micro-dystrophin protein comprising infecting a host cell with the rAAV vector of claim 1, and expressing a functional micro-dystrophin protein in the host cell.

16. A plasmid comprising a micro-dystrophin gene comprising a nucleotide sequence encoding the amino acid sequence set forth in SEQ ID NO: 8.

17. The plasmid of claim 16, further comprising a selectable marker.

18. The plasmid of claim 16, wherein the plasmid further comprises in the 5′ to 3′ direction an inverted terminal repeat (ITR), a muscle-specific control element, a chimeric intron sequence, the micro-dystrophin gene, a poly A tail, and an ITR.

19. The plasmid of claim 16, wherein the AAV vector lacks AAV rep and cap genes.

20. A method of producing a recombinant AAV particle comprising culturing a cell comprising the plasmid of claim 16 and recovering rAAV particles from the supernatant of the cells.

21. The method of claim 20, wherein the cell is a packaging cell.

22. A bacterial cell comprising the plasmid of claim 16.

23. A packaging cell comprising the plasmid of claim 16.

24. The packaging cell of claim 23, wherein the packaging cell comprises HEK 293 cells, MRC-5 cells, WI-38 cells, Vero cells, or FrhL-2 cells.


SEQUENCE LISTING (15 sequences)

SEQ ID NO: 1 - DNA, 5910 bp, Adeno-associated virus
  regulatory 120..526: note = promoter - CMV promotor; regulatory_class = promoter
  misc_feature 927..1087: note = Intron - EF1a intron
  misc_feature 1088..1375: note = shRNA-miR29-c
  misc_feature 1257..1284: note = miR-29c
  misc_feature 1380..1854: note = Intron - EF1a intron
  misc_feature 1896..2091: note = polyA
  source 1..5910: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggggttaaactcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccggggccgatccaccggtctttttcgcaacgggtttgccgccagaacacaggtaagtgccgtgtgtggttcccgcgggcggcgacggggcccgtgcgtcccagcgcacatgttcggcgaggcggggcctgcgagcgcggccaccgagaatcggacgggggtagtctcaagctggccggcctgtttgaatgaggcttcagtactttacagaatcgttgcctgcacatcttggaaacacttgctgggattacttcttcaggttaacccaacagaaggctcgagaaggtatattgctgttgacagtgagcgcaaccgatttcaaatggtgctagagtgaagccacagatgtctagcaccatttgaaatcggttatgcctactgcctcggaattcaaggggctactttaggagcaattatcttgtttactaaaactgaataccttgctatctctttgatacattggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagctagcgctaccggactcagatctcgagctcaagctgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcactagtagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 2 - DNA, 296 bp, synthetic construct
  misc_feature 1..296: note = Synthetic oligonucleotide
  misc_feature 1..8: note = FSE-I cut site
  misc_feature 9..136: note = miR-30 backbone
  misc_feature 139..160: note = miR-29c target (sense) strand
  misc_feature 161..175: note = miR-29c target (sense) strand
  misc_feature 176..199: note = miR-30 stem loop
  misc_feature 200..288: note = miR-29c guide (antisense) strand
  misc_feature 289..296: note = miR-30 backbone
  source 1..296: mol_type = other DNA
ggccggcctgtttgaatgaggcttcagtactttacagaatcgttgcctgcacatcttggaaacacttgctgggattacttcttcaggttaacccaacagaaggctcgagaaggtatattgctgttgacagtgagcgcaaccgatttcaaatggtgctagagtgaagccacagatgtctagcaccatttgaaatcggttatgcctactgcctcggaattcaaggggctactttaggagcaattatcttgtttactaaaactgaataccttgctatctctttgatacattggccggcc

SEQ ID NO: 3 - DNA, 22 bp, synthetic construct
  misc_feature 1..22: note = Synthetic oligonucleotide
  source 1..22: mol_type = other DNA
accgatttcaaatggtgctaga

SEQ ID NO: 4 - DNA, 24 bp, synthetic construct
  misc_feature 1..24: note = Synthetic oligonucleotide
  source 1..24: mol_type = other DNA
tctagcaccatttgaaatcggtta

SEQ ID NO: 5 - DNA, 15 bp, synthetic construct
  misc_feature 1..15: note = Synthetic oligonucleotide
  source 1..15: mol_type = other DNA
gtgaagccacagatg

SEQ ID NO: 6 - RNA, 98 nt, synthetic construct
  misc_feature 1..98: note = Synthetic oligonucleotide
  misc_feature 1..19: note = miR-30 backbone
  misc_feature 20..42: note = miR-29c target (sense) strand
  misc_feature 43..57: note = miR-30 stem loop
  misc_feature 58..81: note = miR-29c guide (antisense) strand
  misc_feature 82..98: note = miR-30 backbone
  source 1..98: mol_type = other RNA
  modified_base 1: mod_base = OTHER; note = Uracil
  modified_base 4: mod_base = OTHER; note = Uracil
  modified_base 6..7: mod_base = OTHER; note = Uracil
  modified_base 13: mod_base = OTHER; note = Uracil
  modified_base 26..28: mod_base = OTHER; note = Uracil
  modified_base 33: mod_base = OTHER; note = Uracil
  modified_base 36: mod_base = OTHER; note = Uracil
  modified_base 39: mod_base = OTHER; note = Uracil
  modified_base 44: mod_base = OTHER; note = Uracil
  modified_base 56: mod_base = OTHER; note = Uracil
  modified_base 58: mod_base = OTHER; note = Uracil
  modified_base 60: mod_base = OTHER; note = Uracil
  modified_base 68..70: mod_base = OTHER; note = Uracil
  modified_base 75: mod_base = OTHER; note = Uracil
  modified_base 79..80: mod_base = OTHER; note = Uracil
  modified_base 82: mod_base = OTHER; note = Uracil
  modified_base 86: mod_base = OTHER; note = Uracil
  modified_base 89: mod_base = OTHER; note = Uracil
  modified_base 93: mod_base = OTHER; note = Uracil
tgctgttgacagtgagcgcaaccgatttcaaatggtgctagagtgaagccacagatgtctagcaccatttgaaatcggttatgcctactgcctcggaa

SEQ ID NO: 7 - DNA, 3581 bp, Homo sapiens
  source 1..3581: mol_type = other DNA
  CDS 1..3579
atgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagc

SEQ ID NO: 8 - protein, 1192 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 9 - DNA, 8409 bp, Adeno-associated virus
  regulatory 236..799: note = promoter - MCK promotor; regulatory_class = promoter
  misc_feature 844..993: note = Intron - Chimeric intron sequence
  misc_feature 1004..4584: note = Human Dystrophin sequence
  misc_feature 4585..4640: note = Poly A tail
  misc_feature 6606..7466: note = Ampicillin resistance
  misc_difference 7621..8240: note = pGEX plasmid backbone with pBR322 origin or replication
  source 1..8409: mol_type = other DNA
gcccaatacgcaaaccgcctctccccgcgcgttggccgattcattaatgcagctggcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctaattatctacgtagccatgtctagacagccactatgggtctaggctgcccatgtaaggaggcaaggcctggggacacccgagatgcctggttataattaacccagacatgtggctgctccccccccccaacacctgctgcctgagcctcacccccaccccggtgcctgggtcttaggctctgtacaccatggaggagaagctcgctctaaaaataaccctgtccctggtgggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaagttccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgatttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaagttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcgtacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgggtttgagtgagctgataccgctcgccgcagccgaacgaccgagcgcagcgagtcagtgagcgaccaagcggaagagc

SEQ ID NO: 10 - DNA, 206 bp, synthetic construct
  misc_feature 1..206: note = Synthetic oligonucleotide
  regulatory 1..206: note = enhancer - MCK enhancer; regulatory_class = enhancer
  source 1..206: mol_type = other DNA
cagccactatgggtctaggctgcccatgtaaggaggcaaggcctggggacacccgagatgcctggttataattaacccagacatgtggctgctccccccccccaacacctgctgcctgagcctcacccccaccccggtgcctgggtcttaggctctgtacaccatggaggagaagctcgctctaaaaataaccctgtccctggtgg

SEQ ID NO: 11 - DNA, 358 bp, synthetic construct
  misc_feature 1..358: note = Synthetic oligonucleotide
  regulatory 1..358: note = promoter - MCK promoter; regulatory_class = promoter
  source 1..358: mol_type = other DNA
gctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 12 - DNA, 5920 bp, Adeno-associated virus
  misc_feature 1..5920: note = pAAV.MCK.miR26C Sequence
  misc_difference 4..9: note = n is a, c, g, t or u
  regulatory 190..395: note = enhancer - MCK enhancer; regulatory_class = enhancer
  regulatory 396..753: note = promoter - MCK promoter; regulatory_class = promoter
  misc_feature 1316..1608: note = shRNA-miR29-c with primary seed sequence
  misc_feature 1487..1512: note = miR-29c
  misc_feature 2094..2146: note = SV40 PolyA
  misc_difference 2326..2331: note = n is a, c, g, t or u
  source 1..5920: mol_type = other DNA
ctgnnnnnngcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctaattatctacgtagccatgtctagacagccactatgggtctaggctgcccatgtaaggaggcaaggcctggggacacccgagatgcctggttataattaacccagacatgtggctgctccccccccccaacacctgctgcctgagcctcacccccaccccggtgcctgggtcttaggctctgtacaccatggaggagaagctcgctctaaaaataaccctgtccctggtgggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccggggccgatccaccggtctttttcgcaacgggtttgccgccagaacacaggtaagtgccgtgtgtggttcccgcgggcggcgacggggcccgtgcgtcccagcgcacatgttcggcgaggcggggcctgcgagcgcggccaccgagaatcggacgggggtagtctcaagctggccggcctgtttgaatgaggcttcagtactttacagaatcgttgcctgcacatcttggaaacacttgctgggattacttcttcaggttaacccaacagaaggctcgagaaggtatattgctgttgacagtgagcgcaaccgatttcaaatggtgctagagtgaagccacagatgtctagcaccatttgaaatcggttatgcctactgcctcggaattcaaggggctactttaggagcaattatcttgtttactaaaactgaataccttgctatctctttgatacattggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagctagtgcggccgcaataaaagatctttattttcattagatctgtgtgttggttttttgtgtgtctagacatggctacgtagataattagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcnnnnnncagctggcgtaatagcgaagaggcccgcaccgatcgcccttcccaacagttgcgcagcctgaatggcgaatggaagttccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgatttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaagttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcgtacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgggtttgagtgagctgataccgctcgccgcagccgaacgaccgagcgcagcgagtcagtgagcgaccaagcggaagagc

SEQ ID NO: 13 - RNA, 88 nt, synthetic construct
  source 1..88: mol_type = other RNA
atctcttacacaggctgaccgatttctcctggtgttcagagtctgtttttgtctagcaccatttgaaatcggttatgatgtaggggga

SEQ ID NO: 14 - RNA, 71 nt, synthetic construct
  source 1..71: mol_type = other RNA
gcgactgtaaacatcctcgactggaagctgtgaagccacaaatgggctttcagtcggatgtttgcagctgc

SEQ ID NO: 15 - RNA, 97 nt, synthetic construct
  source 1..97: mol_type = other RNA
tgctgttgacagtgagcgcccgcctgaagtctctgattaatagtgaagccacagatgtattaatcagagacttcaggcggttgcctactgcctcgga